(19) United States
(12) Patent Application Publication
(10) Pub. No.: US 2024/0376499 A1
(43) Pub. Date: Nov. 14, 2024

(54) MODULAR PRIME EDITING SYSTEMS FOR GENOME ENGINEERING

(71) Applicant: UNIVERSITY OF MASSACHUSETTS, Boston, MA (US)

(72) Inventors: Bin Liu, Boston, MA (US); Erik Sontheimer, Auburndale, MA (US); Wen Xue, Natick, MA (US); Xiaolong Dong, Worcester, MA (US)

(21) Appl. No.: 18/577,623

(22) PCT Filed: Jul. 6, 2022
(86) PCT No.: PCT/US22/36230

§ 371 Date: Jan. 8, 2024

Related U.S. Application Data
(60) Provisional application No. 63/220,303, filed on Jul. 9, 2021.
(60) Provisional application No. 63/273,622, filed on Oct. 29, 2021.
(60) Provisional application No. 63/327,129, filed on Apr. 4, 2022.

(51) Int. Cl. C12N 15/90; C12N 9/22; C12N 15/11

(52) U.S. Cl. CPC C12N 15/907; C12N 9/22; C12N 15/11; C12N 2310/20

(57) ABSTRACT

A modular prime editing (sPE) system is disclosed with separate and independent Cas9 nickase and reverse transcriptase template. This sPE system results in precise and efficient genome editing in cells and in adult mouse liver which is advantageous over conventional fusion proteins. Additionally, a pegRNA can be separated into an sgRNA and a prime editor template RNA, designated as a petRNA. This flexible, and modular system, is an improvement in the art to obtain precise genome editing.

### STATEMENT OF GOVERNMENT INTEREST

This invention was made with government support under HL137167, HL131471 and HL147367 awarded by The National Institutes of Health. The government has certain rights in the invention.

### FIELD OF THE INVENTION

The present invention relates to the field of genomic engineering. In particular, a modular prime editing (sPE) system is disclosed comprising elements including, but not limited to, a Cas9 nickase (nCas9) ribonucleic acid (RNA), a prime editor guide RNA (pegRNA), a prime editor template RNA (petRNA), an sgRNA or a reverse transcriptase template (RTT) RNA, such that both the nCas9 RNA and the RTT RNA are free and independent molecules. This modular sPE composition results in precise and efficient genome editing in cells and in adult mouse liver which is advantageous over conventional split PE fusion constructs. This flexible and modular system is an improvement in the art to obtain precise genome editing.

### BACKGROUND

Correction of genetic mutations in vivo has broad potential therapeutic application for a range of human genetic diseases. Prime editors (PE) composed of a Cas9 nickase fused to an engineered reverse transcriptase have enabled precise nucleotide changes, sequence insertions and deletions. Anzalone et al., “Search-and-replace genome editing without double-strand breaks or donor DNA” Nature 576:149-157 (2019).

This innovative technology does not induce double-stranded DNA breaks and does not require a donor DNA template in conjunction with homology directed repair to introduce precise sequence changes into the genome. The ability to precisely install or correct pathogenic mutations makes prime editors an excellent tool to perform somatic genome editing.

Unlike base editing systems, prime editors can introduce any nucleotide substitution as well as insertions and deletions, and do not suffer from the challenges of bystander base conversion. These abilities may provide important advantages in some sequence contexts. Prime editor consists of a Cas9 nickase (H840A)-reverse transcriptase (RT) fusion protein paired with a pegRNA with desired edits. However, the potential of clinical use of PE is hampered by the large size for delivery (total length>6.3 kb).

What is needed in the art is a prime editor system having a configuration more compatible with adeno-associated virus and RNA-based delivery platforms.

### SUMMARY OF THE INVENTION

The present invention relates to the field of genomic engineering. In particular, a modular prime editing (sPE) system is disclosed comprising components including, but not limited to, a Cas9 nickase (nCas9) ribonucleic acid (RNA), a prime editor guide RNA (pegRNA), an sgRNA, a prime editor template RNA (petRNA), or a reverse transcriptase template (RTT) RNA, such that both the nCas9 sgRNA and the RTT RNA are free and independent molecules. This modular sPE composition results in precise and efficient genome editing in cells and in adult mouse liver which is advantageous over conventional split PE fusion constructs. This flexible, and modular system, is an improvement in the art to obtain precise genome editing.

In one embodiment, the present invention contemplates a modular prime editing system comprising: i) a first ribonucleic acid (RNA) encoding a Cas9 nickase protein and a reverse transcriptase protein; and ii) a second RNA encoding a component selected from the group consisting of a single guide RNA, a primer binding site and a reverse transcriptase template (RTT), wherein said first RNA and said second RNA are not attached or tethered. In one embodiment, the second RNA comprises a prime editor guide RNA (pegRNA). In one embodiment, the second RNA comprises a prime editor template RNA (petRNA). In one embodiment, the petRNA is circularized. In one embodiment, the petRNA is linear. In one embodiment, the first RNA is less than 4.5 kB. In one embodiment, the second RNA is less than 4.5 kB. In one embodiment, the combination of the first and the second RNA is less then 4.5 kB.

In one embodiment, the present invention contemplates a composition, comprising: a) a ribonucleic acid (RNA) delivery system; and b) a modular prime editing system comprising: i) a first ribonucleic acid (RNA) encoding a Cas9 nickase protein and a reverse transcriptase protein; and ii) a second RNA comprising a component selected from the group consisting of a single guide RNA, a primer binding site and a reverse transcriptase template (RTT), wherein said first RNA and said second RNA are not attached or tethered. In one embodiment, the second RNA comprises a prime editor guide RNA (pegRNA). In one embodiment, the second RNA comprises a prime editor template RNA (petRNA). In one embodiment, the petRNA is circularized. In one embodiment, the petRNA is linear. In one embodiment, the first RNA is less than 4.5 kB. In one embodiment, the second RNA is less than 4.5 kB. In one embodiment, the combination of the first and the second RNA is less than 4.5 kB. In one embodiment, the RNA delivery system is an adeno-associated virus (AAV) delivery system. In one embodiment, the first RNA and the second RNA is encased within the AAV. In one embodiment, the RNA delivery system is a ribonucleoprotein delivery system. In one embodiment, the RNA delivery system is a microparticle system. In one embodiment, the RNA delivery system is a liposome system.

In one embodiment, the present invention contemplates a method, comprising; a) providing; i) a patient expressing at least one symptom of a genetic disease or disorder; and ii) a pharmaceutically acceptable composition comprising an RNA delivery system and a modular prime editing system comprising a first ribonucleic acid (RNA) comprising a Cas9 nickase protein and a reverse transcriptase protein, and a second RNA encoding a component selected from the group consisting of a single guide RNA, a primer binding site and a reverse transcriptase template (RTT), wherein said first RNA and said second RNA are not attached or tethered; b) administering the pharmaceutically acceptable composition to said patient, wherein said at least one symptom of a genetic disease or disorder is reduced. In one embodiment, the administering further comprises expressing the first RNA and the second RNA. In one embodiment, the administering further comprises translating said encoded Cas9 nickase protein. In one embodiment, the administering further comprises translating said encoded reverse transcriptase protein. In one embodiment, the second RNA comprises a prime editor template RNA (petRNA). In one embodiment, the petRNA is circularized. In one embodiment, the petRNA is linear. In one embodiment, the first RNA is less than 4.5 kB. In one embodiment, the second RNA is less than 4.5 kB. In one embodiment, the combination of the first and the second RNA is less than 4.5 kB. In one embodiment, the RNA delivery system comprises an adeno-associated virus system. In one embodiment, the RNA delivery system is a ribonucleoprotein delivery system. In one embodiment, the RNA delivery system is a microparticle system. In one embodiment, the RNA delivery system is a liposome system. In one embodiment, the single guide RNA hybridizes to a gene including, but not limited to, a HBB gene, a HEXA gene, a PSEN1 gene, a PRNP gene, an IDS gene and an IDUA gene.

In one embodiment, the present invention contemplates a method, comprising: a) providing; i) an ribonucleic acid (RNA) molecule comprising an aptamer sequence, a primer binding site sequence and a reverse transcription template sequence; ii) a first linker sequence attached to the aptamer sequence; iii) a second linker sequence attached to the primer binding site sequence; iv) a first group I catalytic intron sequence attached to the 3′ end of the first linker sequence; and v) a second group I catalytic intron sequence attached to the 5′ end of the second linker sequence; b) self-catalyzing the first and second group I intron sequence in the presence of guanosine triphosphate wherein the first linker sequence is attached to the second linker sequence to create a circular RNA molecule. In one embodiment, the aptamer is a PP7 aptamer. In one embodiment, the aptamer is an MS2 aptamer. In one embodiment, the RNA molecule is a prime editing RNA molecule. In one embodiment, the circular RNA molecule is a prime editing template RNA molecule (petRNA).

In one embodiment, the present invention contemplates a circular ribonucleic acid (RNA) molecule comprising: i) an aptamer sequence comprising a 3′ linker sequence; and ii) a primer binding site comprising a 5′ linker sequence, wherein the 3′ linker sequence is attached to the 5′ linker sequence. In one embodiment, the aptamer is a PP7 aptamer. In one embodiment, the aptamer is an MS2 aptamer. In one embodiment, the circular RNA molecule further comprises a reverse transcriptase template sequence. In one embodiment, the RNA molecule is a prime editing RNA molecule. In one embodiment, the circular RNA molecule is a prime editing template RNA molecule (petRNA).

### Definitions

To facilitate the understanding of this invention, a number of terms are defined below. Terms defined herein have meanings as commonly understood by a person of ordinary skill in the areas relevant to the present invention. Terms such as “a” “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

The term “catalytically impaired Cas9 nickase” or “nCas9”, as used herein refers to a mutated Cas9 which renders the nuclease able to cleave only one strand of deoxyribonucleic acid backbone. Depending on the position of the mutation within the Cas9 protein sequence either the target or non-target strand is cleaved. In the case of a prime editor the non-target strand is selectively cleaved.

The term “engineered reverse transcriptase” as used herein, refers to a protein that converts RNA into DNA and contains specific mutations that effect its activity efficiency. One example, of a reverse transcriptase is a Moloney murine leukemia virus reverse transcriptase (M-MLV RT).

The term “reverse transcriptase template” as used herein refers to a ribonucleic acid sequence that is utilized as a substrate for a reverse transcriptase protein that is part of the fusion protein complex as contemplated herein. Such templates provide the necessary information to edit a DNA sequence to support conversions including, but not limited to, base conversions, sequence insertions or sequence deletions.

The term “primer binding site” as used herein, refers to a specific nucleic acid sequence within the pegRNA that is complementary to the 3′ end of the nicked DNA strand. This allows annealing of the free 3′ end of the genomic DNA for extension by the reverse transcriptase based on the template sequence encoded in the pegRNA.

The term, “prime editing guide RNA molecule” or “pegRNA molecule” as used herein, refers to a Cas9 guide RNA molecule that encodes the crRNA-tracrRNA fused to a primer binding site (PBS) and a reverse transcriptase template (RTT) nucleic acid sequence. The primer binding site hybridizes to a desired genomic sequence released by the binding and cleavage of the Cas9 nickase. The 3′ end of the genomic sequence is extended by the reverse transcriptase based on the reverse transcriptase template sequence.

The term, “petRNA molecule”, as used herein, refers to an RNA molecule that encodes a primer binding site (PBS) and a reverse transcriptase template (RTT). The petRNA may also encode stem loops. The petRNA may also be linear or circularized.

The term “editing” or “gene editing” as used herein, refers to a genetic manipulation of a DNA sequence. Such a manipulation includes, but is not limited to, a base conversion, a sequence insertion and/or a sequence deletion.

The term “group I catalytic intron” as used herein, refers to large self-splicing ribozymes which self-catalyze an excision from ribonucleotides including, but not limited to, mRNA, tRNA and rRNA. See, FIG. 19. Nielsen et al., “Group I introns: Moving in new directions” RNA Biol. 6(4):375-83 (2009); and Cech T., “Self-splicing of group I introns” Annu. Rev. Biochem. 59:543-568 (1990). Their core secondary structure included paired regions. Woodson S, “Structure and assembly of group I introns” Curr. Opin. Struct. Biol. 15(3):324-330 (2005). These paired regions self-assembly into domains: i) the P4-P6 domain formed from stacking of P5, P4, P6 and P6a helices; and ii) the P3-P9 domain formed from the P8, P3, P7 and P9 helices). Cate et al., “Crystal structure of a group I ribozyme domain: principles of RNA packing”. Science. 273 (5282):1678-1685 (1996). Group I introns often have long open reading frames inserted in loop regions.

The term “prime editing” as used herein, is a genome editing technology by which the genome of living organisms may be modified. Prime editing manipulates the genetic information of a targeted DNA site to essentially “rewrite” the coded sequences.

The term “prime editor” or “PE” as used herein, is a fusion protein comprising a catalytically impaired Cas9 endonuclease that can nick DNA and is fused to an engineered reverse transcriptase enzyme and attached to a prime editing guide RNA (pegRNA). The pegRNA is capable of programming the nCas9 to recognize a target site with the encoded crRNA-tracrRNA (as does a conventional single guide RNA). The resulting nicked genomic DNA can be extended by the reverse transcriptase based on the pegRNA template sequence to contain a new sequence. Once one strand is recoded, cellular DNA repair pathways can cause conversion of the local DNA sequence to match the new sequence. Such manipulation includes, but is not limited to, insertions, deletions, and base-to-base conversions without the need for double strand breaks (DSBs) or donor DNA templates. For example, such prime editing may be performed by a Cas9 CRISPR platform programmed with a pegRNA, such as a catalytically impaired Cas9 nickase platform with an appropriate reverse transcriptase.

The term “conversion” as used herein, refers to any manipulation of a nucleic acid sequence that converts a mutated sequence into a wild type sequence, or a wild type sequence into a mutated sequence. For example, a converted sequence includes, but is not limited to, a base pair conversion, a nucleic acid sequence insertion or a nucleic acid sequence deletion.

The term “editing-related indels” as used herein, refers to the generation of off-target and/or unintended nucleotide sequence insertions created by a prime editor.

The term “split-intein prime editor protein” refers to a prime editor protein that has been split into amino-terminal (PE2-N) and carboxy-terminal (PE2-C) segments, which are then fused into a full length PE by a trans-splicing intein. This configuration imparts flexibility to the prime editor thereby facilitating a packaging into an adeno-associated virus (AAV).

As used herein, the term “CRISPRs” or “Clustered Regularly Interspaced Short Palindromic Repeats” refers to an acronym for DNA loci that contain multiple, short, direct repetitions of base sequences. Each repetition contains a series of bases followed by 30 or so base pairs known as “spacer” sequence. The spacers are short segments of DNA from a virus and may serve as a ‘memory’ of past exposures to facilitate an adaptive defense against future invasions. Doudna et al. Genome editing. The new frontier of genome engineering with CRISPR-Cas9” Science 346(6213):1258096 (2014).

As used herein, the term “Cas” or “CRISPR-associated (cas)” refers to genes often associated with CRISPR repeat-spacer arrays.

As used herein, the term “Cas9” refers to a nuclease from type II CRISPR systems, an enzyme specialized for generating double-strand breaks in DNA, with two active cutting sites (the HNH and RuvC domains), one for each strand of the double helix. tracrRNA and spacer RNA may be combined into a “single-guide RNA” (sgRNA) molecule that, mixed with Cas9, could find and cleave DNA targets through Watson-Crick pairing between the guide sequence within the sgRNA and the target DNA sequence, Jinek et al. A programmable dual-RNA-guided DNA endonuclease in adaptive bacterial immunity” Science 337(6096):816-821 (2012).

As used herein, the term “catalytically active Cas9” refers to an unmodified Cas9 nuclease comprising full nuclease activity.

The term “nickase” as used herein, refers to a nuclease that cleaves only a single DNA strand, either due to its natural function or because it has been engineered to cleave only a single DNA strand. Cas9 nickase variants that have either the RuvC or the HNH domain mutated provide control over which DNA strand is cleaved and which remains intact. Jinek et al., “A programmable dual-RNA-guided DNA endonuclease in adaptive bacterial immunity” Science 337(6096):816-821 (2012) and Cong et al. Multiplex genome engineering using CRISPR/Cas systems” Science 339(6121):819-823 (2013).

The term, “trans-activating crRNA”, “tracrRNA” as used herein, refers to a small trans-encoded RNA. For example, CRISPR/Cas (clustered, regularly interspaced short palindromic repeats/CRISPR-associated proteins) constitutes an RNA-mediated defense system, which protects against viruses and plasmids. This defensive pathway has three steps. First a copy of the invading nucleic acid is integrated into the CRISPR locus. Next, CRISPR RNAs (crRNAs) are transcribed from this CRISPR locus. The crRNAs are then incorporated into construct complexes, where the crRNA guides the complex to the invading nucleic acid and the Cas proteins degrade this nucleic acid. There are several pathways of CRISPR activation, one of which requires a tracrRNA, which plays a role in the maturation of crRNA. TracrRNA is complementary to the repeat sequence of the pre-crRNA, forming an RNA duplex. This is cleaved by RNase III, an RNA-specific ribonuclease, to form a crRNA/tracrRNA hybrid. This hybrid acts as a guide for the endonuclease Cas9, which cleaves the invading nucleic acid.

The term “protospacer adjacent motif” (or PAM) as used herein, refers to a DNA sequence that may be required for a Cas9/sgRNA to form an R-loop to interrogate a specific DNA sequence through Watson-Crick pairing of its guide RNA with the genome. The PAM specificity may be a function of the DNA-binding specificity of the Cas9 protein (e.g., a “protospacer adjacent motif recognition domain” at the C-terminus of Cas9).

The terms “protospacer adjacent motif recognition domain”, “PAM Interacting Domain” or “PID” as used herein, refers to a Cas9 amino acid sequence that comprises a binding site to a DNA target PAM sequence.

The term “binding site” as used herein, refers to any molecular arrangement having a specific tertiary and/or quaternary structure that undergoes a physical attachment or close association with a binding component. For example, the molecular arrangement may comprise a sequence of amino acids. Alternatively, the molecular arrangement may comprise a sequence a nucleic acids. Furthermore, the molecular arrangement may comprise a lipid bilayer or other biological material.

As used herein, the term “sgRNA” refers to single guide RNA used in conjunction with CRISPR associated systems (Cas). sgRNAs are a fusion of crRNA and tracrRNA and contain nucleotides of sequence complementary to the desired target site. Jinek et al., “A programmable dual-RNA-guided DNA endonuclease in adaptive bacterial immunity” Science 337(6096):816-821 (2012) Watson-Crick pairing of the sgRNA with the target site permits R-loop formation, which in conjunction with a functional PAM permits DNA cleavage or in the case of nuclease-deficient Cas9 allows binds to the DNA at that locus.

As used herein, the term “orthogonal” refers to targets that are non-overlapping, uncorrelated, or independent. For example, if two orthogonal Cas9 isoforms were utilized, they would employ orthogonal sgRNAs that only program one of the Cas9 isoforms for DNA recognition and cleavage. Esvelt et al., “Orthogonal Cas9 proteins for RNA-guided gene regulation and editing” Nat Methods 10(11):1116-1121 (2013). For example, this would allow one Cas9 isoform (e.g. S. pyogenes Cas9 or SpyCas9) to function as a nuclease programmed by a sgRNA that may be specific to it, and another Cas9 isoform (e.g. N. meningitidis Cas9 or NmeCas9) to operate as a nuclease-dead Cas9 that provides DNA targeting to a binding site through its PAM specificity and orthogonal sgRNA. Other Cas9s include S. aureus Cas9 or SauCas9 and A. naeslundii Cas9 or AnaCas9.

The term “truncated” as used herein, when used in reference to either a polynucleotide sequence or an amino acid sequence means that at least a portion of the wild type sequence may be absent. In some cases, truncated guide sequences within the sgRNA or crRNA may improve the editing precision of Cas9. Fu, et al. “Improving CRISPR-Cas nuclease specificity using truncated guide RNAs” Nat Biotechnol. 2014 March; 32(3):279-284 (2014).

The term “base pairs” as used herein, refer to specific nucleobases (also termed nitrogenous bases), that are the building blocks of nucleotide sequences that form a primary structure of both DNA and RNA. Double-stranded DNA may be characterized by specific hydrogen bonding patterns. Base pairs may include, but are not limited to, guanine-cytosine and adenine-thymine base pairs.

The term “specific genomic target” as used herein, refers to any pre-determined nucleotide sequence capable of binding to a Cas9 protein contemplated herein. The target may include, but may be not limited to, a nucleotide sequence complementary to a programmable DNA binding domain or an orthogonal Cas9 protein programmed with its own guide RNA, a nucleotide sequence complementary to a single guide RNA, a protospacer adjacent motif recognition sequence, an on-target binding sequence and an off-target binding sequence.

As used herein, the term “edit” “editing” or “edited” refers to a method of altering a nucleic acid sequence of a polynucleotide (e.g., for example, a wild type naturally occurring nucleic acid sequence or a mutated naturally occurring sequence) by selective deletion of a specific genomic target or the specific inclusion of new sequence through the use of an exogenously supplied DNA template. Such a specific genomic target includes, but may be not limited to, a chromosomal region, mitochondrial DNA, a gene, a promoter, an open reading frame or any nucleic acid sequence.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “associated with” or “linked to” as used herein, refers to an art-accepted causal relationship between a genetic mutation and a medical condition or disease. For example, it is art-accepted that a patient having an HTT gene comprising a tandem CAG repeat expansion mutation has, or is a risk for, Huntington's disease.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “derived from” as used herein, refers to the source of a sample, a compound or a sequence. In one respect, a sample, a compound or a sequence may be derived from an organism or particular species. In another respect, a sample, a compound or sequence may be derived from a larger complex or sequence.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “polypeptide”, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens or larger.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable and to refer to a sequence of amino acids.

The term “portion” when used in reference to a nucleotide sequence refers to fragments of that nucleotide sequence. The fragments may range in size from 5 nucleotide residues to the entire nucleotide sequence minus one nucleic acid residue. When used in reference to an amino acid sequence refers to fragments of that amino acid sequence. The fragment may range in size from 2 amino acid residues to the entire amino acid sequence minus one amino acid residue.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxy-ribonucleotide. The primer must be sufficiently long to prime the synthesis of extension products in the presence of the inducing agent. The exact lengths of the primers will depend on many factors, including temperature, source of primer and the use of the method.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “an oligonucleotide having a nucleotide sequence encoding a gene” means a nucleic acid sequence comprising the coding region of a gene, i.e. the nucleic acid sequence which encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

### BRIEF DESCRIPTION OF THE FIGURES

FIG. 1 presents exemplary embodiments of MS2-PE2 and SunTag-PE2 fusion protein constructs.

FIG. 1A: Schematic overview of MS2-PE2. The MS2 coat protein (MCP) was fused to the N-terminus of M-MLV reverse transcriptase to enable recruitment by the MS2-pegRNAs.

FIG. 1B: Representative sizes of Cas9 nickase and MCP-RT ORFs.

FIG. 1C: Engineered MS2-pegRNAs with MS2 sequences appended into distinct sgRNA stem-loops, or onto the 3′ terminus.

FIG. 1D: Schematic overview of SunTag-PE2.

FIG. 1E: Schematics of scFv-RT and GCN4-Cas9 nickase. The scFv was fused to the N terminus of M-MLV RT (top). The 10×GCN4 epitope was fused to either the N terminus (SunTag-PE) or the C terminus of SpyCas9H840A (PE-SunTag).

FIG. 2 presents exemplary data showing Split-PE, SunTag-PE3 and MS2-PE3 tested in mCherry reporter line and an endogenous locus. Data are derived from three or more independent experiments (mean±s.d.) unless otherwise indicated.

FIG. 2A: A diagram of an mCherry reporter line that functions by converting a premature stop codon.

FIG. 2B: Sequences of RTT and PBS, non-cognate (PBS+RTT), non-cognate PBS, and non-cognate RTT for the mCherry reporter line.

FIG. 2C: Multiple MS2-pegRNAs tested in mCherry reporter cell lines. The pegRNA with MS2 on the repeat/anti-repeat stem-loop (pegRNA-1.1) has the highest editing efficiency (higher even than that of a PE3 construct) in this mCherry reporter line. The pegRNA1.1-Cas9H840A-MCP-RT system is designated hereinafter as MS2-PE3.

FIG. 2D: SunTag-PE3 and PE3-SunTag were tested in the mCherry reporter cell line.

FIG. 2E: Sanger sequencing and EditR quantification of PE3, Split PE, SunTag-PE3 and MS2-PE3 by installing “CTT” at HEK3 sites in HEK293T cells. All plasmids were transfected with the same molar ratio using Lipofectamine™ 3000 Transfection Reagent. Genomic DNAs were isolated 72 h post transfection.

FIG. 2F: Dose dependence of the RT-encoding plasmid. One microgram of H840A plasmid was cotransfected with plasmids encoding additional modular sPE components (pegRNA (0.3 g), nicking sgRNA (0.1 g), and RT (0.01-2 g)) per well.

FIG. 3 presents exemplary data of amplicon sequencing of MS2-PE3 and SunTag-PE3 at multiple endogenous sites. Data are derived from three or more independent experiments (mean s.d.) unless otherwise indicated.

FIG. 3A: MS2-PE3 for editing by 1-bp substitution at multiple endogenous loci, including HEK3, RNF2, VEGFA, and FANCF in HEK293T cells.

FIG. 3B: SunTag-PE3 for RNF2 and VEGFR editing to generate a 1-bp substitution in HEK293T cells.

FIG. 4 presents exemplary data of SunTag-PE3 and MS2-PE3 constructs tested in reporter lines. Data are derived from three or more independent experiments (mean±s.d.) unless otherwise indicated.

FIG. 4A: A diagram of the GFP reporter line that is activated by precise insertion of 18 bp (in place of a 39-bp non-functional sequence). Indels (+1) can restore mCherry expression.

FIG. 4B: A diagram of the GFP reporter line that is activated by deletion of 47 bp; indels (+1) can restore mCherry expression.

FIG. 4C: MS2-PE3 was tested in the GFP reporter line shown in FIG. 4A.

FIG. 4D: SunTag-PE3 was tested in the GFP reporter line shown in FIG. 4A.

FIG. 4E: MS2-PE3 was tested in the GFP reporter line shown in FIG. 4B.

FIG. 5 presents exemplary data comparing genome editing data in cells with split PE2 fusion constructs (PE2) and a modular sPE system. Nicking sgRNAs (PE3 format) were used for all prime editing. Data are derived from three or more independent experiments (mean±s.d.) unless otherwise indicated.

FIG. 5A: A conventional split prime editor (PE2) comprising a fusion protein of a Cas9 nickase (nCas9) fused to a reverse transcriptase and a presently disclosed modular prime editor (sPE) that is expressed from two separate vectors.

FIG. 5B: A split PE2 fusion construct without inteins which is too large to integrate into an AAV (e.g., >4.5 kB), as compared to a modular sPE system which is configured to integrate into an AAV (both vectors<4.5 kB).

FIG. 5C: mCherry reporter cells transfected with the indicated plasmids.

FIG. 5D: Prime editing of a GFP reporter line by deletion of 47 bp for restoring GFP expression using a PE3 fusion construct or a modular sPE. Indels (+1) can restore mCherry expression.

FIG. 5E: Amplicon sequencing of a PE3 fusion construct and a modular sPE for multiple endogenous sites in 293T cells.

FIG. 5F: Prime editing by alternative RT orthologs at the FANCF site by 3-nt transversions (+2 C to T and +4-5 TG to AC). Human codon-optimized E.r. maturase RT and GsI-IIC RT were cloned into a split-PE2 construct in place of the M-MLV RT (FL=full-length; split=untethered nCas9 and RT).

FIG. 6 presents exemplary data showing split prime editor 2 by alternative reverse transcriptases. Data are derived from three or more independent experiments (mean±s.d.) unless otherwise indicated.

FIG. 6A: Illustration of split prime editors with alternative RTs. Human codon-optimized E.r. maturase RT and GsI-IIC RT were cloned into the split-PE2 construct in place of the M-MLV RT.

FIG. 6B: Prime editing by alternative RT orthologs at the VEGFA site by 3-nt transversions (+2 G to C and +4-5 GG to CT). FL=full length; split=untethered nCas9 and RT.

FIG. 7 presents exemplary data showing prime editing activity using a mutant PE2 fusion construct and a modular sPE system with representative Sanger sequencing traces. HEK293T cells were transfected with indicated plasmids, along with others encoding pegRNA and nicking sgRNA. Prime editing introduces a 3-nt transversion at the FANCF locus (+2 C to T and +4-5 TG to AC). The experiment was repeated two times. Sanger sequencing traces were analyzed by EditR.

FIG. 8 presents exemplary data comparing split-PE3 fusion constructs and modular sPE systems that result in genome editing in adult mice.

FIG. 8A: Prime editor-induced oncogenic activation of Ctnnb1 by a 3-bp in-frame deletion. Split-PE3 fusion constructs (n=4) and modular sPE systems (n=5) were delivered by hydrodynamic tail-vein injection along with the MYC transposon and transposase plasmids.

FIG. 8B: Tumor numbers in livers were counted 25 days after injection with split-PE3 fusion constructs or modular sPE systems (mean±s.d.).

FIG. 8C: Representative images of tumors in liver with split-PE3 fusion constructs or modular sPE systems. Control group was pegRNA-injected only.

FIG. 8D: Immunohistochemistry staining shows nuclear beta-Catenin in liver tumors. Scale bars: 100 m (20×lens).

FIG. 8E: Amplicon sequencing from representative animals using genomic DNA isolated from tumors.

FIG. 9 illustrates prime editing by separate RNA modules. Plasmids expressing RNAs were co-transfected with Cas9H840A and a modular RT RNA, which lacks the MCP domain. Nicking sgRNAs were used for all prime editing. Data are derived from three or more independent experiments (mean±s.d.) unless otherwise indicated.

FIG. 9A: Schematic of proof-of-concept experiment on delivering the RT template separately from the pegRNA. The 3′ extension of the pegRNA e.g., the RTT-PBS sequence) was removed from the 3′ of the tracrRNA scaffold and provided separately under the control of a U6 promoter. An sgRNA plasmid was co-transfected to carry out the nicking event in conjunction with the nCas9.

FIG. 9B: Illustration of the circularization pathway to generate petRNAs.

FIG. 9C: PE efficiency by modular RNA components at the FANCF locus introducing a 3-nt transversion (+2 C to T and +4-5 TG to AC).

FIG. 10 illustrates prime editing by modular RNA components. Nicking sgRNAs are used for all prime editing. Data are derived from three or more independent experiments (mean s.d.) unless otherwise indicated.

FIG. 10A: Illustration of a prime editing template RNA (petRNA). The RTT-PBS sequence was arranged to the 3′ end of an MS2 hairpin in an independent RNA, with and/or without circularization via a ribozyme-enabled pathway to give rise to a circular petRNA and/or a linear petRNA. A 3′ MS2 version of the petRNA (petRNA-3′) was also tested. The petRNA works in lieu of the pegRNA at the nick site to recruit an MCP-fused RT and serves as the template for reverse transcription.

FIG. 10B: A model of petRNA activity in a modular sPE system.

FIG. 10C: PE efficiency of petRNA at the FANCF locus. Plasmids expressing the pegRNA or petRNA-sgRNA were co-transfected with nCas9 and MCP-RT. PE introduces a 3-nt transversion (+2 C to T and +4-5 TG to AC).

FIG. 10D: Validation of petRNA at the DNMT1 locus (+5 G to T).

FIG. 11 presents exemplary data showing an adaptability validation of petRNA designs. Data are derived from three or more independent experiments (mean±s.d.) unless otherwise indicated.

FIG. 11A: Validation of petRNA design at VEGFA, RUNX1 and EMX1 loci. VEGFA: +2 G to C and +4-5 GG to CT. RUNX1: +5 G to T. EMX1: +5 G to T. Data are based on four independent experiments.

FIG. 11B: Validation of petRNA adaptability to an alternative nickase. The petRNA was designed to target a site at the FANCF locus where SpyCas9 and SauCas9 nickases share the same nick and thus a single petRNA guide/primer/template sequence. The petRNA and the MCP-RT were co-transfected with plasmids encoding SpyCas9H840A-sgRNA or SauCas9N580A. Nicking sgRNAs were used for all prime editing.

FIG. 12 presents exemplary data of amplicon sequencing results showing prime editing efficiency of sPE compared to PE3 at FANCF, VEGFA and HEK3 loci in HEK293T cells. Nicking guide was used in all experiments. N=3.

FIGS. 12A-E: Conventional mRNA delivery.

FIG. 12F: Ribonucleofection (RNP) delivery

FIG. 13 present exemplary data showing FAH gene editing data after in vivo administration of a sPE dual-AAV prime editor composition in mice.

FIG. 13A: Schematic of the sPE dual-AAV prime editor. Cas9H840A and M-MLV RT were subcloned into two AAV8 vectors.

FIG. 13B: Schematic of the in vivo experiments. The dual AAV8 sPE (1×10¹² vg total) was delivered to six-week-old Fah mutant mice via tail-vein injection.

FIG. 13C: Immunohistochemistry staining and Hematoxylin and Eosin staining (H&E) (n=3 mice, 24 days without NTBC).

FIG. 13D: Dual AAV rescued body weight loss after NTBC withdrawal. Body weight ratio is normalized to day 0 of NTBC withdrawal. Mouse 1-3 are three mice treated with dual AAV. NC:non-treated control.

FIG. 13E: Amplicon sequencing from representative animals (IHC) using genomic DNA isolated from livers. FIG. 14 presents exemplary data showing that when modular sPE is delivered as circular mRNAs comparable editing outcomes are achieved as those rendered by traditional linear mRNAs.

FIG. 14A: A circularized mRNA construct that is capless, unmodified and covalently enclosed with an IRES sequence for in-cell translation. Wesselhoeft et al.

FIG. 14B: A representative diagram depicting engineered sPE circRNAs with Cas9^{H840A} and RT as separate coding regions.

FIG. 14C: Efficacy of circRNA-delivered sPE effectors in HEK293T cells. The precise editing outcomes (3 nt transversion at the VEGFA locus) were assessed by ddPCR. Both synthesized pegRNA and nicking guide were used in all experiments. 1=PE3; and 2-5=modular sPEs. N=1.

FIG. 15 presents exemplary data of a Northern Blot analysis demonstrating the integrity and abundance of pegRNAs and petRNAs after 72 h post-transfection of a dual AAV modular sPE transfection into HEK293T cells.

FIG. 16 presents exemplary data of amplicon sequencing results showing the efficiency of prime editing by modular sPE, pegRNA or petRNA at various endogenous loci in HEK293T cells after transient plasmid transfection with co-transfection of H840A and MCP-RT plasmids. For petRNA experiments, the sgRNA with same spacer sequence as the pegRNA counterpart was co-transfected. Nicking guide was used in all experiments. N=3.

FIG. 16A: Editing efficiency of modular sPE compared to PE3 at disease-relevant locus HBB.

FIG. 16B: Editing efficiency of modular sPE compared to PE3 at disease-relevant locus HEXA.

FIG. 16C: Editing efficiency of modular sPE compared to PE3 at disease-relevant locus PSEN1.

FIG. 16D: Comparison of editing efficiencies by pegRNA and petNRA at disease-relevant locus PRNP.

FIG. 16E: Comparison of editing efficiencies by pegRNA and petNRA at disease-relevant locus IDS.

FIG. 16F: Comparison of editing efficiencies by pegRNA and petNRA at disease-relevant locus IDUA.

FIG. 17 presents an exemplary suite of linear prime editing template RNAs (linpetRNAs) that enable efficient precision editing.

FIG. 17A: Composition of a linpetRNA. The MS2-RTT-PBS sequence (green) is joined by a structural motif (cyan) at the 3′ end for increased stability.

FIG. 17B: Diagram showing the putative mechanism underlying linpetRNA functionality in an sPE system in which sgRNA, MCP-RT and nCas9H840A are used.

FIG. 17C: Precision editing at the FANCF locus introducing a 3-nt substitution using linpetRNAs expressed from U6-driven plasmids. Editing efficiency was determined by amplicon sequencing.

FIG. 17D: Proof-of-concept experiment showing efficient editing at the FANCF locus using a synthetic linpetRNA containing a 3′ mpknot pseudoknot motif. The sgRNA and nicking sgRNA were provided as synthetic RNAs and the MCP-RT and nCas9H840A were provided as mRNAs. Editing efficiency was determined by ddPCR.

FIG. 17E: Illustration showing PP7-PCP affinity as a good alternative to MS2-MCP tethering for circular and linear petRNAs. A PP7 aptamer was used in lieu of the MS2 aptamer for petRNA and linpetRNA designs, to function in tandem with RT with an N-terminal PCP or 2×PCP fusion.

FIG. 17F: Proof-of-concept experiment showing successful precision editing using PP7-PCP-tethered petRNA and linpetRNAs. Editing efficiency was determined by ddPCR.

FIG. 18 presents an exemplary method of prime editing template RNA (petRNA) in vitro mass production with group I intron splicing.

FIG. 18A: Illustration of a catalytic pathway for in vitro petRNA production. A MS2-RTT-PBS sequence (green) is flanked by permutated Anabaena group I intron sequences (orange) via linker sequences (grey). A free GTP initiates a nucleophilic attack at a 5′ intron splice site, which is followed by a second catalytic step by a 3′ linker hydroxyl group at the 3′ intron splice site, circularizing a MS2-RTT-PBS sequence and freeing two intron fragments. The petRNA is then enriched by HPLC purification.

FIG. 18B: Diagram depicting the putative mechanism underlying petRNA functionality in an sPE system in which sgRNA, MCP-RT and nCas9H840A are used.

FIG. 18C: Proof-of-concept experiments showing efficient precision editing at the FANCF locus introducing a 3-nt substitution and at the HEK3 locus inserting a 40 nt LoxP sequence using corresponding in vitro-made petRNAs. Editing efficiency was determined by ddPCR.

FIG. 19 presents one embodiment of a group I catalytic intron.

### DETAILED DESCRIPTION OF THE INVENTION

The present invention relates to the field of genomic engineering. In particular, a modular prime editing (sPE) system is disclosed comprising elements including, but not limited to, a Cas9 nickase (nCas9) ribonucleic acid (RNA), a prime editor guide RNA (pegRNA), a prime editor template RNA (petRNA), an sgRNA or a reverse transcriptase template (RTT) RNA, such that both the nCas9 RNA and the RTT RNA are free and independent molecules. This modular sPE composition results in precise and efficient genome editing in cells and in adult mouse liver which is advantageous over conventional split PE fusion constructs. This flexible, and modular system, is an improvement in the art to obtain precise genome editing.

In one embodiment, the present invention contemplates a modular PE system that results in versatile and efficient somatic genome editing. The data presented herein shows that a modular prime editor (sPE) system has comparable gene editing with a conventional PE2 and/or split PE2/3 fusion constructs. The modular sPE system offers advantages over these known split PE constructs, including its configuration to permit AAV delivery. In one embodiment, a modular sPE system integrates into a dual-AAV in which sufficient space remains to permit accommodation of additional modifications or control elements including, but not limited to, a modified and controllable RT, and/or an engineered or multiplexed sgRNAs, pegRNAs or petRNAs. In addition, reduction in the molecular size of either a Cas9 nickase or an RTT, as compared to a split-PE2 fusion construct may improve the yields of mRNA and/or protein production for nonviral delivery.

The data presented herein also indicates that free and independent RT components can successfully engage a RNA-DNA hybrid at a Cas9 nicking site without a direct fusion to Cas9. Although it is not necessary to understand the mechanism of an invention, it is believed that an untethered RT is recruited to a RNA-DNA hybrid at a nick created by a pegRNA. Despite recent reports addressing PE off-target effects, it is further believed that tethered or untethered RT could process endogenous RNA-RNA or RNA-DNA hybrids and induce potential genomic integration events. Jin et al., Genome-wide specificity of prime editors in plants. Nat Biotechnol, 319 (2021); and Zhang, L. et al. Reverse-transcribed SARS-CoV-2 RNA can integrate into the genome of cultured human cells and can be expressed in patient-derived tissues. Proc Natl Acad Sci USA 118 (2021).

In one embodiment, use of a prime editing template ribonucleic acid (petRNA) allows programmable, high efficiency editing at a given nicking site; when delivered as a cocktail of tiled petRNAs. In one embodiment, the petRNA comprises a primer binding site. In one embodiment, the petRNA comprises an RTT. In one embodiment, the petRNA is circular. In one embodiment, the petRNA is linear. As used herein, these petRNAs are referred to as an sPE system which comprises a plurality of engineered RNAs. In on embodiment, a circular pet RNA contains an RTT-PBS sequence next to an MS2 aptamer which can be covalently enclosed in-cell by an endogenous RtcB ligase. The affinity of the aptamer to the MS2-coating protein (MCP) fused to the MMLV RT facilitates efficient prime editing in mammalian cells in a petRNA-mediated fashion, in lieu of a conventionally implemented pegRNA approach.

### I. Conventional Prime Editor Systems

Prime editors enable deletion, insertion, and base substitution without double-strand breaks. Anzalone et al., “Search-and-replace genome editing without double-strand breaks or donor DNA” Nature 576:149-157 (2019). However, this known fusion of a Cas9 nickase (nCas9; PE2) and a Moloney murine leukemia virus reverse transcriptase (M-MLV RT)) is >6.3 kb. This size is beyond the packaging capacity of a single adeno-associated virus (AAV).

Production of such a large protein in recombinant form in high yield to accommodate ribonucleoprotein (RNP) delivery can also be challenging. Some split Cas9 fusion construct strategies have been tested for the delivery of genome editing tools, including split inteins and MS2 or SunTag tethers. However, most of those split Cas9 fusion construct approaches have not yet been applied to prime editors. Wang et al., “CRISPR-Based Therapeutic Genome Editing: Strategies and In Vivo Delivery by AAV Vectors” Cell 181:136-150 (2020): Truong et al., “Development of an intein-mediated split-Cas9 system for gene therapy” Nucleic Acids Res 43:6450-6458 (2015); Maji et al., “Multidimensional chemical control of CRISPR-Cas9” Nat Chem Biol 13:9-11 (2017)” Liu et al., “A chemical-inducible CRISPR-Cas9 system for rapid control of genome editing” Nat Chem Biol 12:980-987 (2016): Li et al., “SWISS: multiplexed orthogonal genome editing in plants with a Cas9 nickase and engineered CRISPR RNA scaffolds” Genome Biol 21:141 (2020): Konermann et al., “Genome-scale transcriptional activation by an engineered CRISPR-Cas9 complex” Nature 517:583-588 (2015)” Wang et al., “sgBE: a structure-guided design of sgRNA architecture specifies base editing window and enables simultaneous conversion of cytosine and adenosine” Genome Biol 21:222 (2020); Jiang et al., “BE-PLUS: a new base editing tool with broadened editing window and enhanced fidelity” Cell Res 28:855-861 (2018).

These previously reported PE systems may also include a conjugated RNA that consists of a single guide RNA (sgRNA), a 3′ extension containing the RT template (RTT) nucleotide and a primer binding site (PBS), referred to herein as a prime editor sgRNA (e.g., pegRNA). Despite their usefulness, such pegRNAs are prone to misfolding due to inevitable inappropriate base pairing between the PBS and a spacer, as well as potential RTT-scaffold binding interactions. Finally, the 3′-terminal extension in the pegRNA is exposed to the cytosol and is therefore susceptible to degradation by nucleases, which may compromise the integrity of the pegRNA. Therefore, efforts to reduce pegRNA misfolding and instability are needed.

### II. Conventional Split Prime Editor Constructs

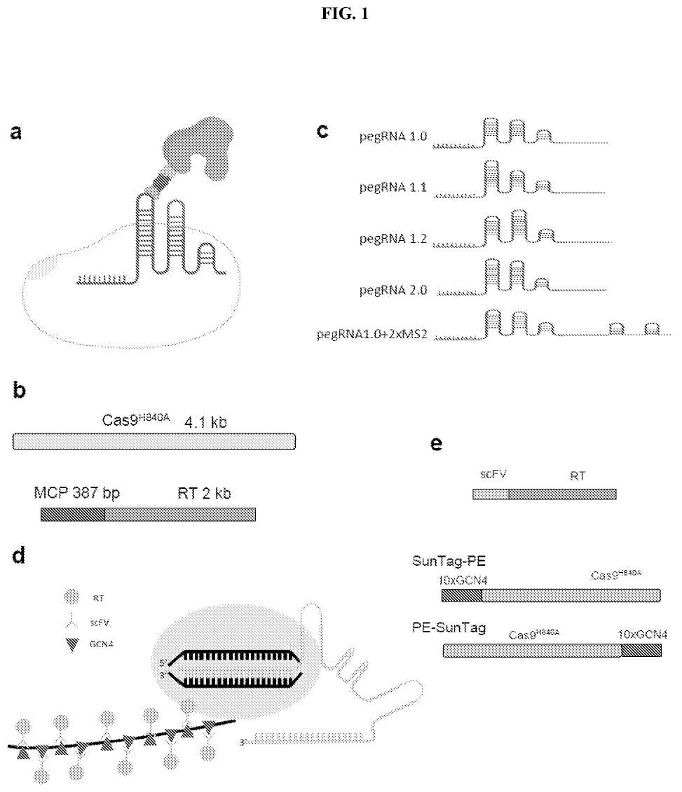

Previously reported split prime editor fusion constructs include, but are not limited to, an MS2-PE2 and SunTag-PE2 fusion constructs. See, FIGS. 1A and 1D. MS2-PE2 comprises an MS2 coat protein (MCP) fused to the N-terminus of M-MLV RT. See, FIG. 1B. Multiple MS2-pegRNAs were engineered by incorporating MS2 stem-loops into different positions of the sgRNA. See, FIG. 1C. Additionally, a split SunTag fusion construct was created by fusing an scFv to the N-terminus of M-MLV RT. See, FIGS. 1D and 1E. Subsequently, the SunTag scFv-RT fusion construct was recruited by either GCN4-nCas9 or nCas9-GCN4. These two PE 2 fusion constructs are designated herein as SunTag-PE2 (GCN4-nCas9) and PE2-SunTag (nCas9-GCN4) based on domain order. Both MS2- and SunTag-based split-PE fusion constructs were compared with a presently disclosed modular PE system (herein referred to as “sPE”) where the nCas9 and RTT are not attached or linked with tethers, such that the PE protein and the RTT-PBS components are free and independent. See, FIGS. 5A and 5B.

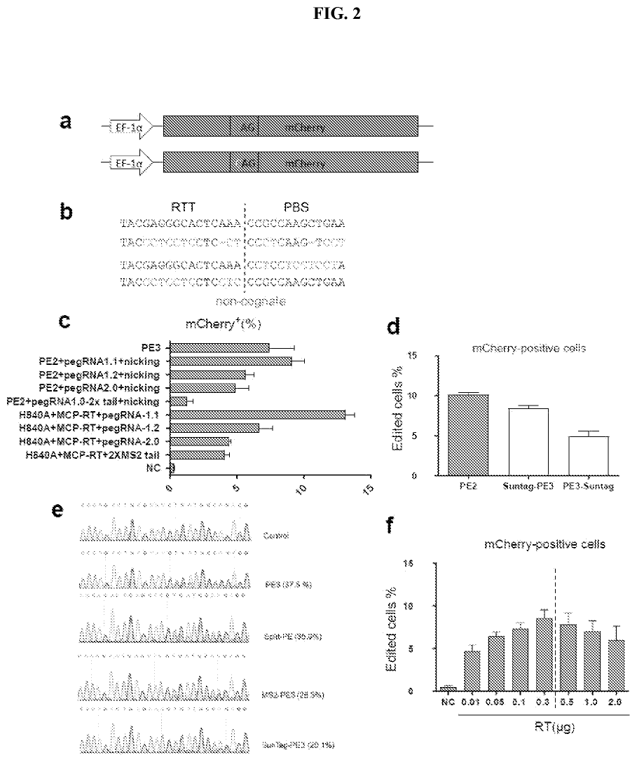

The data presented herein of the MS2, SunTag and sPE platforms were used in a prime editor 3 (PE3) format. The PE3 format differs from PE2 by inclusion of an additional sgRNA that directs nicking of the unedited strand, thereby biasing repair. The respective nCas9-, RT-, pegRNA-, and nicking sgRNA-expressing plasmids were co-transfected into a HEK293T-derived mCherry reporter lentivector-transduced cell line with a premature TAG stop codon that can be reverted to wild type codon, yielding a red fluorescence signal. Liu et al., “Improved prime editors enable pathogenic allele correction and cancer modelling in adult mice” Nat Commun 12:2121 (2021). See, FIGS. 2A and 2B. The most potent MS2- and SunTag-tethered configurations were comparable in editing efficiency to a PE3 fusion construct. See, FIGS. 2C and 2D, cf FIG. 5C.

### B. Modular PE Systems

In one embodiment, a modular sPE system comprises an sgRNA. In one embodiment, the sgRNA binds to a distant target site on a non-edited strand such that the Cas9 nuclease nicks the non-edited strand, to bias the repair process. This provides an advantage over a “conventional” PE system, which includes an nCas9-RT fusion protein and a pegRNA, with or without an additional sgRNA to steer repair to the desired strand.

Surprisingly, modular sPE3 systems generated edited (mCherry) cells with similar efficiency as the fused (PE3) and tethered (MS2-PE3-1.1 and SunTag-PE3) systems. See, FIG. 5C. These results were confirmed with a separate set of pegRNAs that drive a 3-nt insertion in an endogenous locus (HEK3). Sanger sequencing revealed that modular sPE3 gene editing efficiency was again very similar to that of PE3 and higher than those of MS2-PE3-1.1 and SunTag-PE3. See, FIG. 2. A titration of the RT plasmid indicated that it can be transfected at lower levels with little effect on efficiency with the mCherry reporter.

### See, FIG. 2F.

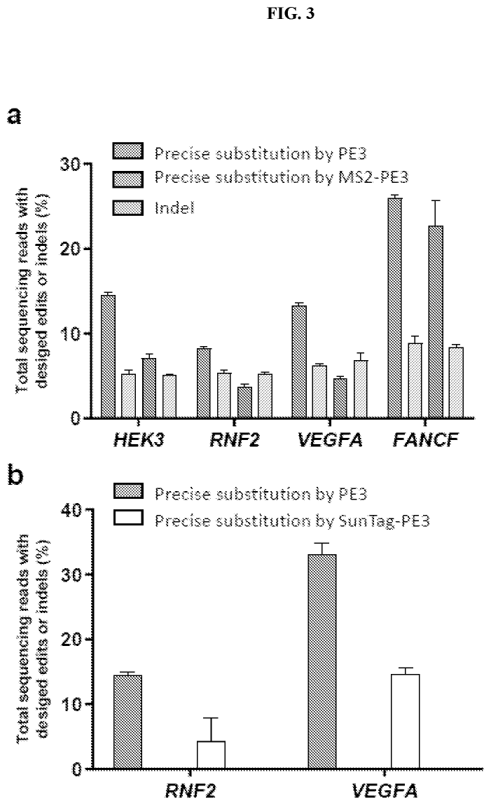

To test the efficiencies of MS2-PE3-1.1, SunTag-PE3, and modular sPE with larger insertions or deletions, they were tested alongside PE3 with two “traffic light reporters” in which GFP is activated by repair of a disruption sequence (an 18-bp replacement of a 39-bp insertion, or the removal of a 47-bp insertion). See, FIGS. 3A and 3B. The data shows that the modular sPE system was just as efficient as a PE3 fusion construct with the precise 47-bp deletion. See, FIG. 5D. Further, both MS2-PE3 and SunTag-PE3 fusion constructs were nearly as efficient as a PE3 fusion construct with the two reporters. See, FIG. 2B. More comprehensive evaluations of the gene editing efficiency of MS2-PE2 and SunTag-PE2 fusion constructs in different reporter lines and multiple endogenous sites revealed that the overall editing efficiency of MS2-PE2 and SunTag-PE2 is generally lower than a modular sPE system. See, FIG. 3.

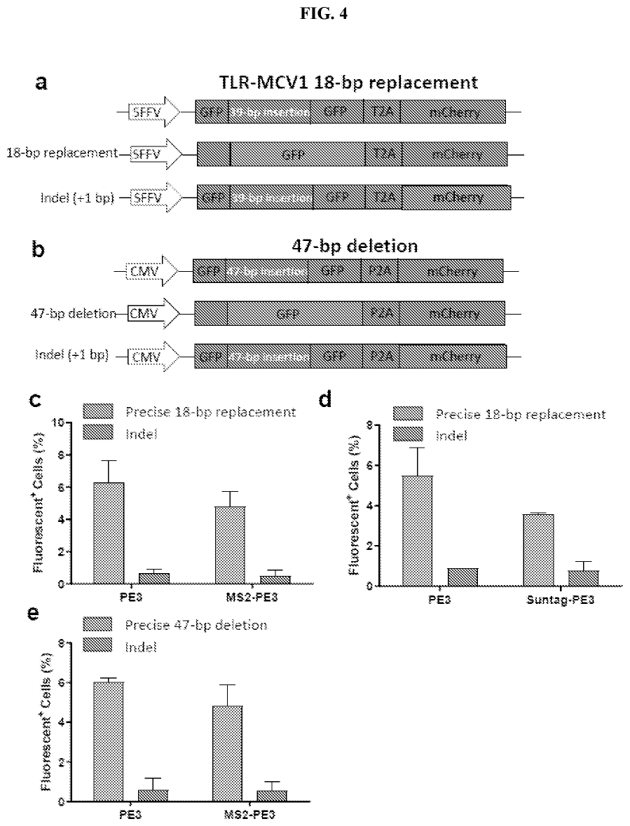

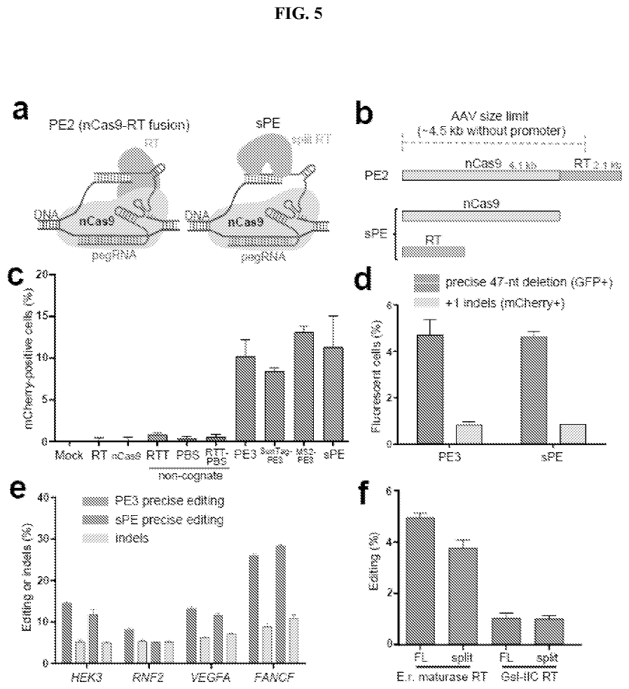

The MS2-PE2 data shows that pegRNA with an MS2 hairpin integrated on the repeat/anti-repeat stem-loop (pegRNA-1.1) has the highest editing efficiency in the mCherry reporter line, and even exceeded that of the original PE2 construct at this targeted site. Incorporation of MS2 in other positions reduced editing efficiency. See, FIGS. 2A and 2C; FIG. 3A. This pegRNA1.1-nCas9-MCP-RT construct is designated herein after as MS2-PE2. Although MS2-PE2 appeared promising with the mCherry reporter line, it did not show consistent gene editing efficiency in the two GFP reporter lines and at multiple endogenous sites by amplicon sequencing. See, FIG. 4.

Although it is not necessary to understand the mechanism of an invention, it is believed that the MS2 sequence may influence the architecture and folding of pegRNA with particular PBS and RTT sequences, which may cause different editing efficiency at distinct sites. Taken together, the data suggest that MS2-PE2 is functional for prime editing but with variable efficiency. Similarly, SunTag-PE2 and PE2-SunTag were compared in the mCherry reporter line and found that SunTag-PE2 was superior. See, FIG. 2D. Therefore, the SunTag-PE2 construct was tested in a GFP reporter line and at multiple endogenous sites. See, FIG. 4D. Although SunTag-PE2 was functional at all tested sites, it was consistently less efficient than the conventional PE2.

Next, a modular sPE system and a split PE3 fusion construct were compared for gene editing activity at multiple endogenous sites. The data show that the modular sPE system achieved comparable levels of editing as the split PE3 fusion construct (e.g., up to 30%). See, FIG. 5E.

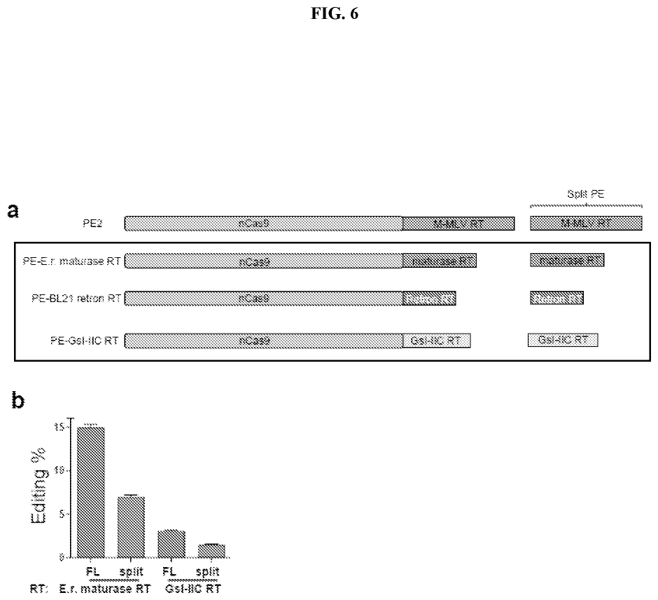

To test the interchangeability of the RT component, the M-MLV RT was replaced in the split PE3 fusion construct and in the modular sPE system with two human codon alternative bacterial RTs E.r. maturase RT (MarathonRT) and GsI-IIC RT (TGIRT III) both of which are compact in size as compared to M-MLV RT. See, FIG. 5F and FIG. 6A. Zhao et al., “An ultraprocessive, accurate reverse transcriptase encoded by a metazoan group II intron” RNA 24:183-195 (2018); Mohr et al., “Thermostable group II intron reverse transcriptase fusion proteins and their use in cDNA synthesis and next-generation RNA sequencing” RNA 19:958-970 (2013): Stamos et al., “Structure of a Thermostable Group II Intron Reverse Transcriptase with Template-Primer and Its Functional and Evolutionary Implications” Mol Cell 68:926-939, e924, (2017).

Prime editors with these alternative RTs successfully worked in tandem with pegRNAs that install 3-nt transversions at FANCF and VEGFA sites, albeit with reduced efficiency compared to a PE2 fusion construct. Notably, these two RT orthologs also mediated gene editing in the modular sPE system with various efficiencies. See, FIG. 5F and FIG. 6B. These results indicate that alternative RTs are applicable to the modular sPE systems. Future efforts to improve thermostability and processivity of RT, facilitated by the modular sPE format, may enable more efficient PE designs.

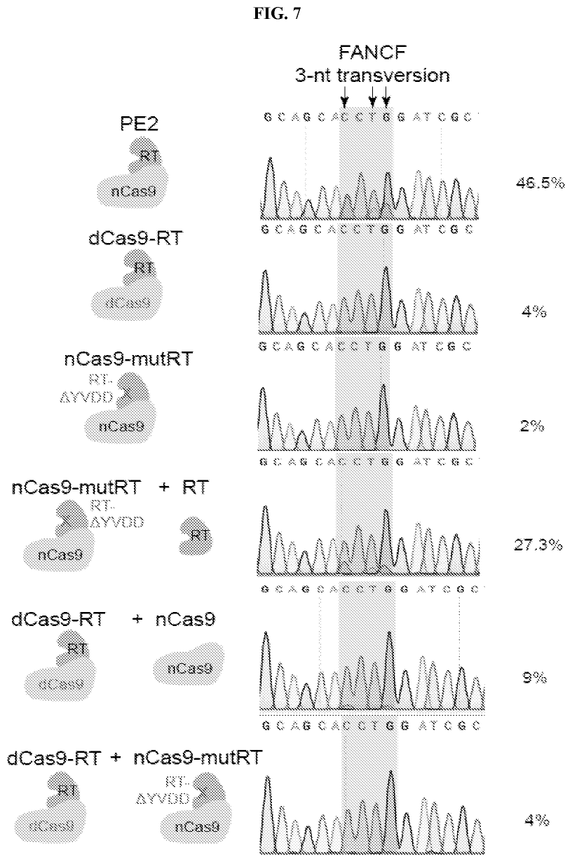

Also generated were two PE2 mutants carrying either Cas9-D10A (dCas9-RT) or RT-ΔYVDD (nCas9-mutRT) mutations. Both constructs were deficient in prime editing at the FANCF locus. Co-transfection of nCas9-mutRT and RT constructs resulted in successful prime editing, whereas co-transfection of nCas9-mutRT and dCas9-RT did not. See, FIG. 7. These data suggest that, in contrast to modular sPE systems, nCas9 and RT derived from separate PE2 proteins do not function in trans at a given nick site, possibly due to local molecular clashes. Together, these data suggested that the modular sPE system has similar capabilities as split PE2 fusion constructs for prime editing in mammalian cells.

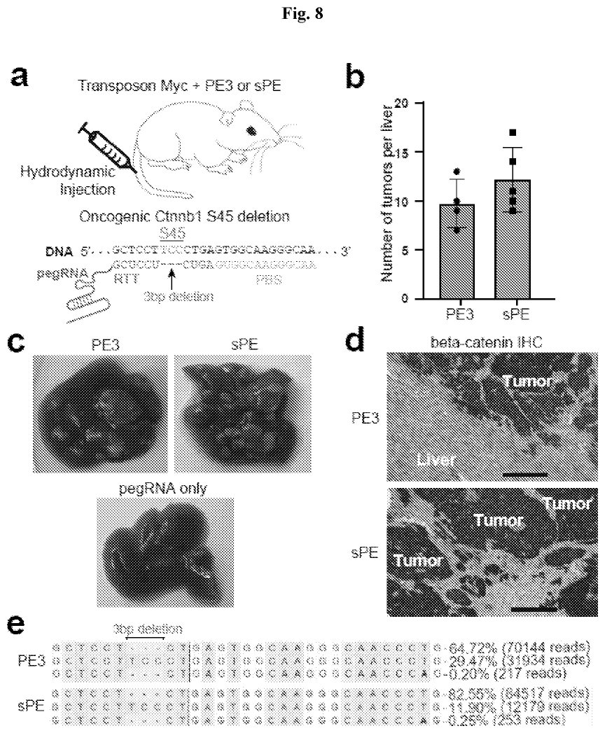

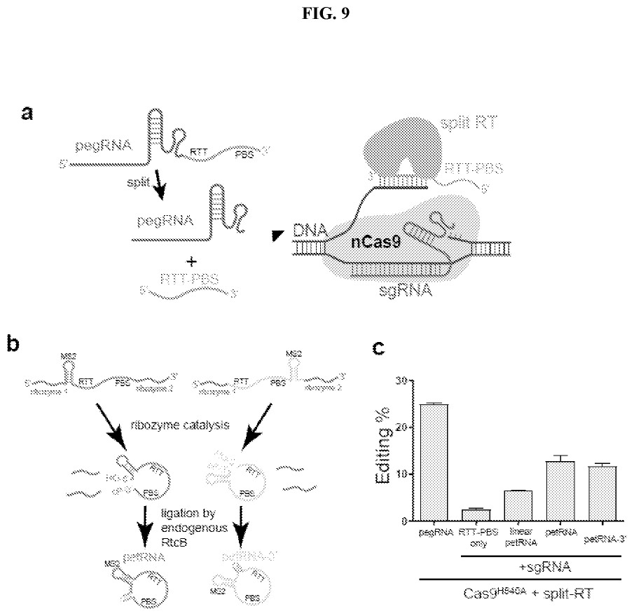

Given the efficient genome editing by modular sPE systems in cells, the modular sPE system was further evaluated in vivo. As precise, microhomology- and HDR-independent deletion is a unique ability of prime editing, the modular sPE system was compared with the split PE2 fusion construct used to delete the S45 codon in the Ctnnb1 (β-catenin) gene to drive tumor formation in adult FVB mice via hydrodynamic tail vein injection. See, FIG. 8A. Four weeks after injection, the split PE2 fusion construct rendered an average of 9.75±2.5 tumors per mouse (n=4), whereas the modular sPE induced 12.2±3.3 tumors per mouse (n=5). See, FIGS. 8B and 8C. Immunohistochemistry staining confirmed the oncogenic activation of β-catenin in the liver tumors and amplicon deep sequencing confirmed the precise 3-bp deletion in Ctnnb1 in both split PE2 fusion constructs and modular sPE system treated groups. See FIGS. 8D and 8E. Therefore, these data show that modular sPE systems result in precise and highly efficient prime editing in vivo.

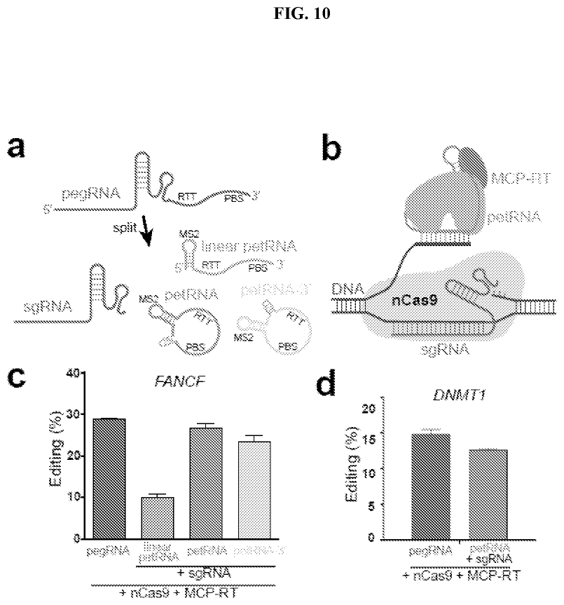

Given the success of the presently disclosed modular sPE systems, wherein the nCas9 and RTT components are untethered and/or not attached, other embodiments comprising a modular RNA component organization were contemplated. See, FIG. 9A. In one embodiment, the modular sPE system comprises a pegRNA split into an sgRNA and a separate RTT-PBS sequence. See, FIG. 10A. An MS2 aptamer was added to the RTT-PBS sequence to tether it to an MCP-RT fusion protein. RTT-PBS was also circularized via a ribozyme- and RtcB108 mediated pathway, referred to herein as a prime editing template RNA (petRNA), and was compared to the linear petRNA. See, FIG. 9B; Litke et al., “Highly efficient expression of circular RNA aptamers in cells using autocatalytic transcripts” Nat Biotechnol 37:667-675 (2019); Roth et al., “A widespread self-cleaving ribozyme class is revealed by bioinformatics.” Nat Chem Biol 10:56-60 (2014).

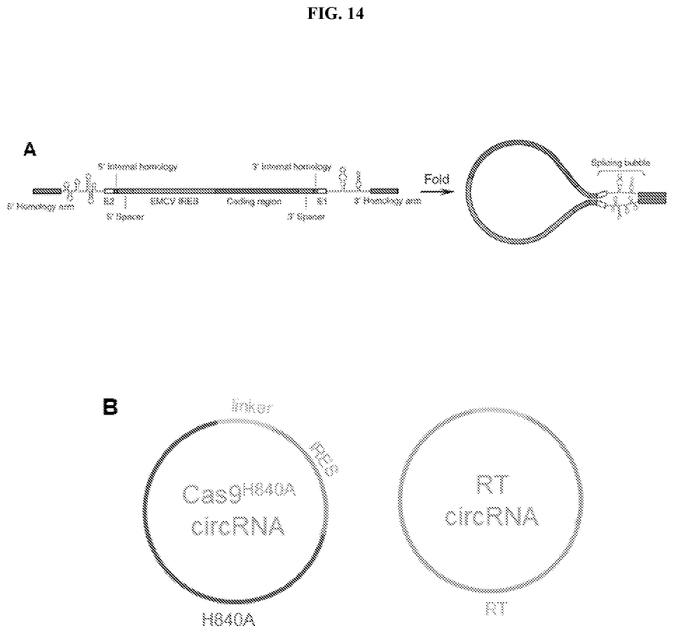

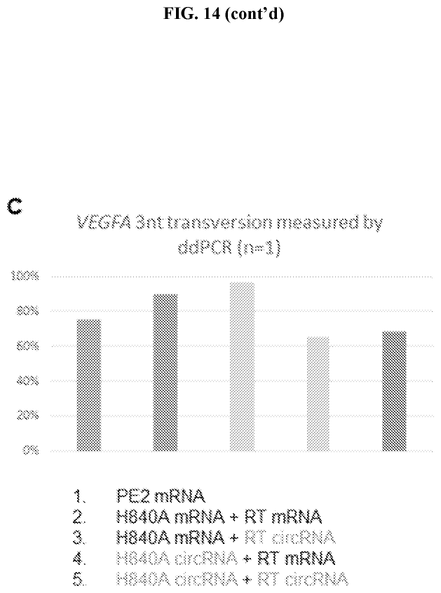

The petRNA and the 3′-MS2 petRNA (petRNA-3′) exhibited comparable gene editing efficiency as that of the pegRNA at the FANCF site. See, FIGS. 10B and 10C. Circularized petRNA was more efficient than the linear petRNA suggesting that stability and abundance of an RTT-PBS RNA is positively correlated with gene editing efficiency. In one embodiment, the present invention contemplates a composition comprising a circular modular sPE RNA comprising a 3′ terminal homology arm and a 5′ terminal homology arm. In one embodiment, the circular modular sPE RNA further comprises a 3′ internal homology arm and a 5′ internal homology arm. In one embodiment, the 3′ and 5′ terminal homology arms are complementary. In one embodiment, the 3′ and 5′ internal homology arms are complementary. In one embodiment, the circular sPE RNA encodes an internal ribosome entry site (IRES). In one embodiment, the circular sPE RNA encodes a coding region. See, FIG. 14A; and Wesselhoeft et al., Nature Communications (2018). In one embodiment, the coding region encodes a Cas9 protein which may, optionally have a H840A mutation. In one embodiment, the coding region encodes a reverse transcriptase protein. See, FIG. 14B. Gene editing efficiency of a 3 nt transversion of VEGFA with a circularized modular sPE RNA was shown to be equivalent to that of a conventional PE3 RNA, whether the circularized modular sPE comprised a coding region encoded either Cas9H840A or an RT, either individually or in combination. See, FIG. 14C.

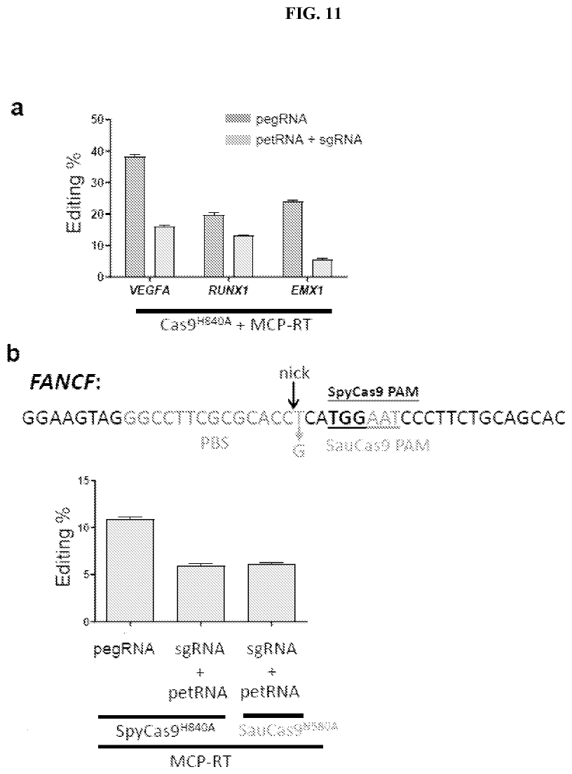

It was also observed that the petRNA supported prime editing activity with unaltered RT, but with an efficiency lower than that observed with the MCP-RT fusion consistent with a benefit from MS2-MCP tethering. See, FIG. 9C. The petRNA design was also validated at the DNMT1 site. See, FIG. 10D. Gene editing by petRNAs was lower compared to pegRNA counterparts at a few other endogenous loci. See, FIG. 11A. These data suggest that petRNA design is an empirical process for specific genomic sites.

To further validate the versatility of a modular sPE system, a site within the FANCF locus was tested where SpyCas9 and SauCas9 target a common spacer sequence. For example, where a shared petRNA could be tested in combination with both SpyCas9 and SauCas9 constructs with their cognate sgRNAs. Both nSpyCas9 and nSauCas9 and their sgRNAs, in combination with MCP-RT and the petRNA, successfully installed a 1-nt transversion. See, FIG. 11B. These results demonstrate that the use of separate RNA modules for nicking and exchange of genetic information also allowed the use of alternative nickases, while maintaining satisfactory gene editing efficiency. The potential use of orthologous Cas9 nickases, or non-CRISPR-Cas constructs (e.g. TALE nickases and ZF nickases), may broaden protospacer accessory motif sequence compatibility away from the traditional NGG sequence and provide more flexibility to prime editing applications. Kim et al., “Precision genome engineering with programmable DNA-nicking enzymes” Genome Res 22:1327-1333 (2012); and Wu et al., “TALE nickase-mediated SP 110 knockin endows cattle with increased resistance to tuberculosis” Proc Natl Acad Sci USA 112:E1530-1539 (2015).

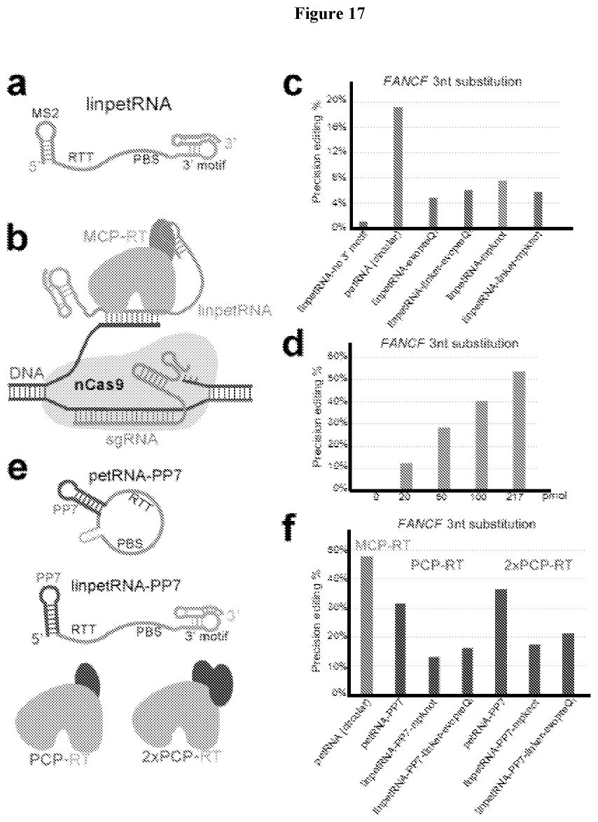

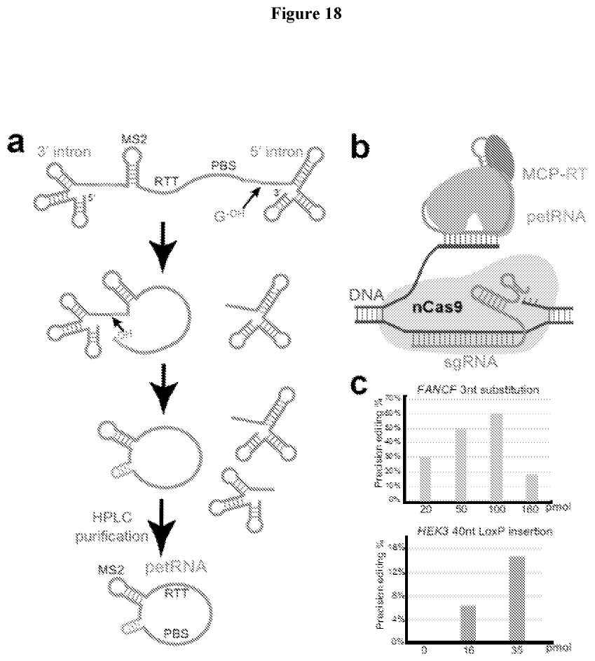

As an alternative tethering embodiment, a PP7 aptamer was constructed in lieu of MS2. FIG. 17E. The data shows that PE systems comprising a PP7-coating protein (PCP)-fused RT proteins (e.g., PP7-petRNAs) performed PE-gene editing successfully. FIG. 17F.

### A. Linear sPE Systems

In one embodiment, the present invention contemplates an sPE system comprising a linear petRNA (linpetRNA). Although it is not necessary to understand the mechanism of an invention, it is believed that conventional solid-state synthesis technologies cannot achieve commerically efficient RNA circularization production levels. As contemplated herein, a nonviral delivery of an sPE system comprising an RNA payload is an advantageous approach for successful in vivo and therapeutical applications. However, preliminary data shows that commercial production efficiency and purification procedures for circular petRNAs would benefit from structural modifications to the construct. Thus, the present invention contemplates alternative petRNA designs and RNA production methods to facilitate the use of petRNAs in mammalian therapeutics.

The data presented herein discloses a linear petRNAs (linpetRNAs) with 3′-conjugated structural motifs to increase RNA stability. FIG. 17A. Mechanistically, linpetRNAs function in the same way as a circular petRNA. FIG. 17B. In fact, the 3′ structural motif of the linpetRNA may elongate the half-life of the RTT-PBS. This improved stability favors an elevated PE efficiency, as indicated by positive effects of similar designs on pegRNAs. Nelson et al., “Engineered pegRNAs improve prime editing efficiency” Nat Biotechnol, doi:10.1038/s41587-021-01039-7 (2021).

U6-driven expression of linpetRNAs were observed to have significantly increased PE activity as compared to a no-3′ motif control. FIG. 17C. The higher gene editing efficiency of circular petRNAs is presumably because they are much more abundant. These data suggested that PE gene editing could be successful using synthetic sgRNA and linpetRNA along with sPE mRNAs. The data presented herein indicates that the synthetic linpetRNA is capable of achieving high PE efficiency when working in tandem with a synthetic sgRNA. FIG. 17D.

Consequently, the production of non-circular RNAs (e.g., linpetRNA) from solid-state syntheses was observed to result in an efficient nonviral sPE delivery. In addition, a linpetRNA design may also be suitable for viral delivery platforms where large RT templates are contemplated, which would be disadvantageous as part of a pegRNA construct.

B. Circularized petRNA Cell-Free Production

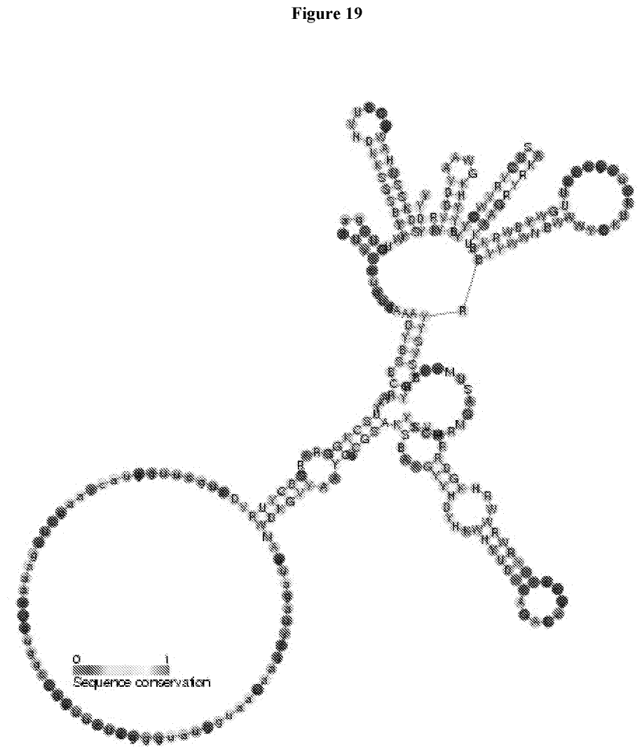

In one embodiment, the present invention contemplates a method for synthesizing a circular petRNA comprising a ligase-free RNA. Although it is not necessary to understand the mechanism of an invention, it is believed that because such a ligase-free method to produce circular petRNAs is an in vitro method, as opposed to a cell-based production method, this would allow a large-scale, low-cost production out of in vitro transcribed (IVT) RNA. In one embodiment, IVT templates (dsDNA generated by PCR) flanked an MS2-RTT-PBS sequence with linker sequences and permutated group I intron fragments. FIG. 18A; Wesselhoeft et al., “Engineering circular RNA for potent and stable translation in eukaryotic cells' Nature Communications 9:2629 (2018); and Wesselhoeft et al., “RNA Circularization Diminishes Immunogenicity and Can Extend Translation Duration In Vivo” Molecular Cell 74, 508-520 (2019).

The group I intron fragments complete a two-step self-catalysis with the addition of GTP and an elevation of temperature. As a result, a linker-flanked MS2-RTT-PBS sequence is covalently enclosed in a petRNA form. FIG. 18A. The data show that ligase-free petRNAs perform similar sPE system gene editing as compared to conventional ligase-containing petRNAs indicated by successful results a low petRNA concentrations. FIG. 18B.

### III. Adeno-Associated Viruses

Adeno-associated viruses (AAV) are small viruses that infect humans and some other primate species. AAVs are small (20 nm) replication-defective, nonenveloped viruses and have linear single-stranded DNA (ssDNA) genome of approximately 4.8 kilobases (kb). Naso et al., “Adeno-Associated Virus (AAV) as a Vector for Gene Therapy” BioDrugs 31(4):317-334 (2017); and Wu et al., “Effect of Genome Size on AAV Vector Packaging” Molecular Therapy 18 (1): 80-86 (2010). AAVs are not currently known to cause disease. The viruses cause a very mild immune response. Several additional features make AAV an attractive candidate for creating viral vectors for gene therapy, and for the creation of isogenic human disease models. Grieger et al., “Adeno-associated Virus as a Gene Therapy Vector: Vector Development, Production and Clinical Applications”; Adeno-associated virus as a gene therapy vector: vector development, production and clinical applications. In: Advances in Biochemical Engineering/Biotechnology. 99. pp. 119-145 (2005). Gene therapy vectors using AAV can infect both dividing and quiescent cells and persist in an extrachromosomal state without integrating into the genome of the host cell, although in the native virus integration of virally carried genes into the host genome does occur. Deyle et al., “Adeno-associated virus vector integration”. Current Opinion in Molecular Therapeutics. 11(4):442-447 (2009).

Development of AAVs as gene therapy vectors eliminated the genomic integration capacity by removal of the rep and cap genes. The modified vector has a promoter to drive transcription of the carried gene which is inserted between inverted terminal repeats (ITRs). AAV-based gene therapy vectors consequently form episomal concatemers in the host cell nucleus. In non-dividing cells, these concatemers remain intact for the life of the host cell. In dividing cells, AAV DNA is lost through cell division, since the episomal DNA is not replicated along with the host cell DNA. Surosky et al., “Adeno-associated virus Rep proteins target DNA sequences to a unique locus in the human genome” Journal of Virology 71(10):7951-7959 (1997).

The AAV genome is built of single-stranded deoxyribonucleic acid (ssDNA), either positive- or negative-sensed, which is about 4.7 kilobase long. The genome comprises ITRs at both ends of the DNA strand, and two open reading frames (ORFs) encoding the rep and cap proteins. The rep ORF is composed of four overlapping genes encoding Rep proteins required for the AAV life cycle. The cap ORF is composed of overlapping nucleotide sequences of capsid proteins (e.g., VP1, VP2 and VP3) which interact to form a capsid with icosahedral symmetry. Carter B J, “Adeno-associated virus and adeno-associated virus vectors for gene delivery”. In: Lassic D D, Templeton N S (eds.). Gene Therapy: Therapeutic Mechanisms and Strategies. New York City: Marcel Dekker, Inc. pp. 41-59 (2000).

AAV inverted terminal repeat (ITR) sequences usually comprise about 145 bases each and are believed required for efficient multiplication of the AAV genome. Bohenzky et al., “Sequence and symmetry requirements within the internal palindromic sequences of the adeno-associated virus terminal repeat” Virology 166(2):316-327 (1988). ITRs also have a hairpin structure which contributes to self-priming that allows a primase-independent synthesis of the second DNA strand. The ITRs were also shown to be required for host cell DNA integration/removal, efficient encapsidation and deoxyribonuclease resistance. Wang et al., “Rescue and replication signals of the adeno-associated virus 2 genome” Journal of Molecular Biology 250(5):573-580 (1995); Weitzman et al., “Adeno-associated virus (AAV) Rep proteins mediate complex formation between AAV DNA and its integration site in human DNA” PNAS USA 91(13): 5808-5812 (1994); and Zhou et al, “In vitro packaging of adeno-associated virus DNA”. Journal of Virology 72(4):3241-3247 (1998). With regard to gene therapy, ITRs are configured in cis next to the therapeutic gene, in contrast the structural (cap) and packaging (rep) proteins which can be delivered in trans. Nony et al., “Novel cis-acting replication element in the adeno-associated virus type 2 genome is involved in amplification of integrated rep-cap sequences” Journal of Virology 75(20):9991-9994 (2001); Nony et al., “Evidence for packaging of rep-cap sequences into adeno-associated virus (AAV) type 2 capsids in the absence of inverted terminal repeats: a model for generation of rep-positive AAV particles” Journal of Virology 77(1):776-781 (2003); Philpott et al., “Efficient integration of recombinant adeno-associated virus DNA vectors requires a p5-rep sequence in cis” Journal of Virology 76(11):5411-5421 (June 2002); and Tullis et al., “Efficient replication of adeno-associated virus type 2 vectors: a cis-acting element outside of the terminal repeats and a minimal size”. Journal of Virology 74(24):11511-11521 (2000).

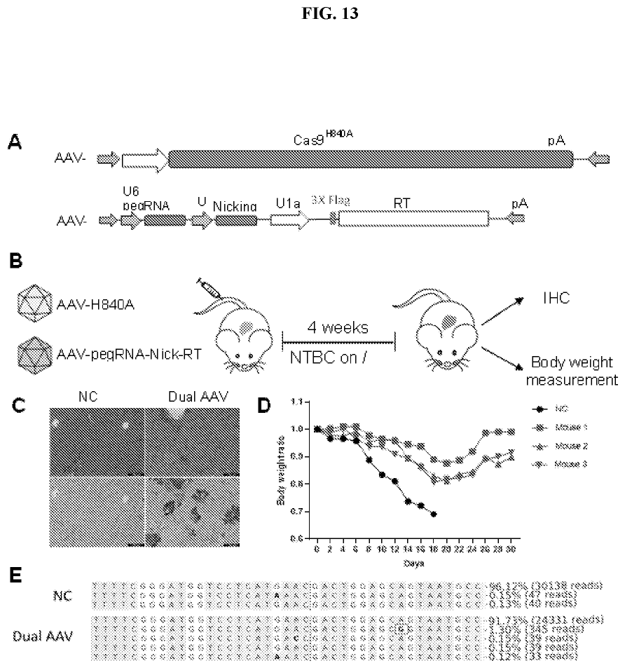

In one embodiment, the present invention contemplates a modular sPE dual-AAV prime editor composition, comprising: i) a first associated adenovirus (AAV) vector encoding a Cas9^{H840A} nuclease protein; and ii) a second AAV vector encoding a reverse transcriptase (RT) protein. See, FIG. 13A. In one embodiment, the second AAV vector further encodes a pegRNA and a nicking sgRNA. In one embodiment, the present invention contemplates a method comprising administering a modular sPE dual-AAV primer editor composition to a patient. See, FIG. 13B. The data show that with in vivo administration of the second AAV vector encoding an M-MLV RT protein, the FAH gene undergoes gene editing of approximately 1.25%. See, FIG. 13C. Co-administration of NTBC was observed to prevent a reduction in body weight. See, FIG. 13D.

### IV. Modular sPE Treatment of Genetic Disease

In one embodiment, the present invention contemplates a method for treating a genetic disease with a composition comprising a modular sPE. For example, administration of a modular sPE to a patient comprising a genetic mutation loci, reverts the mutated loci into a wild type loci through a nucleotide transversion (eg., 1 nt, 2 nt, 3 nt etc., transversion). Such mutated loci may be located on genes including, but not limited to, an HBB gene, a HEXA gene, a PSEN1 gene, a PRNP gene, an IDS gene and/or an IDUA gene.

### A. Hemoglobin Subunit B (HBB) Genetic Diseases

The HBB gene encodes a protein called beta-globin. Beta-globin is a component (subunit) of a larger protein called hemoglobin, which is located inside red blood cells. In adults, hemoglobin normally consists of four protein subunits: two subunits of beta-globin and two subunits of a protein called alpha-globin, which is produced from another gene called HBA. Each of these protein subunits is attached (bound) to an iron-containing molecule called heme; each heme contains an iron molecule in its center that can bind to one oxygen molecule. Hemoglobin within red blood cells binds to oxygen molecules in the lungs. These cells then travel through the bloodstream and deliver oxygen to tissues throughout the body.

### 1. Beta Thalassemia

Nearly 400 mutations in the HBB gene have been found to cause beta thalassemia. Most of the mutations involve a change in a single DNA nucleotide within or near the HBB gene. Other mutations comprise an insertion or deletion of nucleotides in the HBB gene.

HBB gene mutations that decrease beta-globin production result in a condition called beta-plus (β+) thalassemia. Mutations that prevent cells from producing any beta-globin result in beta-zero (β0) thalassemia.

Problems with the subunits that make up hemoglobin, including low levels of beta-globin, reduce or eliminate the production of this molecule. A lack of hemoglobin disrupts the normal development of red blood cells. A shortage of mature red blood cells can reduce the amount of oxygen that is delivered to tissues to below what is needed to satisfy the body's energy needs. A lack of oxygen in the body's tissues can lead to poor growth, organ damage, and other health problems associated with beta thalassemia.

### 2. Methemoglobinemia, Beta-Globin Type

More than 10 mutations in the HBB gene have been found to cause methemoglobinemia, beta-globin type, which is a condition that alters the hemoglobin within red blood cells. These mutations often affect the region of the protein that binds to heme. For hemoglobin to bind to oxygen, the iron within the heme molecule needs to be in a form called ferrous iron (Fe²⁺). The iron within the heme can change to another form of iron called ferric iron (Fe³⁺), which cannot bind to oxygen. Hemoglobin that contains ferric iron is known as methemoglobin and is unable to efficiently deliver oxygen to the body's tissues.

In methemoglobinemia, beta-globin type, mutations in the HBB gene alter the beta-globin protein and promote the heme iron to change from ferrous to ferric. This altered hemoglobin gives the blood a brown color and causes a bluish appearance of the skin, lips, and nails (cyanosis). The signs and symptoms of methemoglobinemia, beta-globin type are generally limited to cyanosis, which does not cause any health problems. However, in rare cases, severe methemoglobinemia, beta-globin type can cause headaches, weakness, and fatigue.

### 3. Sickle Cell Disease

Sickle cell anemia (also called homozygous sickle cell disease or HbSS disease) is the most common form of sickle cell disease. This form is caused by a particular mutation in the HBB gene that results in the production of an abnormal version of beta-globin called hemoglobin S or HbS. In this condition, hemoglobin S replaces both beta-globin subunits in hemoglobin. The mutation that causes hemoglobin S changes a single protein building block (amino acid) in beta-globin. Specifically, the amino acid glutamic acid is replaced with the amino acid valine at position 6 in beta-globin (e.g., Glu6Val or E6V). Replacing glutamic acid with valine causes the abnormal hemoglobin S subunits to stick together and form long, rigid molecules that bend red blood cells into a sickle (crescent) shape. The sickle-shaped cells die prematurely, which can lead to a shortage of red blood cells (anemia). The sickle-shaped cells are rigid and can block small blood vessels, causing severe pain and organ damage.

Mutations in the HBB gene can also cause other abnormalities in beta-globin, leading to other types of sickle cell disease. These abnormal forms of beta-globin are often designated by letters of the alphabet or sometimes by a name. In these other types of sickle cell disease, just one beta-globin subunit is replaced with hemoglobin S. The other beta-globin subunit is replaced with a different abnormal variant, such as hemoglobin C or hemoglobin E.

In hemoglobin SC (HbSC) disease, the beta-globin subunits are replaced by hemoglobin S and hemoglobin C. Hemoglobin C results when the amino acid lysine replaces the amino acid glutamic acid at position 6 in beta-globin (e.g., Glu6Lys or E6K). The severity of hemoglobin SC disease is variable, but it can be as severe as sickle cell anemia. Hemoglobin E (HbE) is caused when the amino acid glutamic acid is replaced with the amino acid lysine at position 26 in beta-globin (written Glu26Lys or E26K). In some cases, the hemoglobin E mutation is present with hemoglobin S. In these cases, a person may have more severe signs and symptoms associated with sickle cell anemia, such as episodes of pain, anemia, and abnormal spleen function.

Other conditions, known as hemoglobin sickle-beta thalassemias (HbSBetaThal), are caused when mutations that produce hemoglobin S and beta thalassemia occur together. Mutations that combine sickle cell disease with beta-zero (β0) thalassemia lead to severe disease, while sickle cell disease combined with beta-plus (β+) thalassemia is generally milder.

### 4. Other HBB Diseases

Hundreds of variations have been identified in the HBB gene. These changes result in the production of different versions of beta-globin. Some of these variations cause no noticeable signs or symptoms and are found when blood work is done for other reasons, while other HBB gene variations may affect a person's health. Two of the most common variants are hemoglobin C and hemoglobin E.

Hemoglobin C (HbC), caused by the Glu6Lys mutation in beta-globin, is more common in people of West African descent than in other populations. People who have two hemoglobin C subunits in their hemoglobin, instead of normal beta-globin, have a mild condition called hemoglobin C disease. This condition often causes chronic anemia, in which the red blood cells are broken down prematurely.

Hemoglobin E (HbE), caused by the Glu26Lys mutation in beta-globin, is a variant of hemoglobin most commonly found in the Southeast Asian population. When a person has two hemoglobin E subunits in their hemoglobin in place of beta-globin, a mild anemia called hemoglobin E disease can occur. In some cases, the mutations that produce hemoglobin E and beta thalassemia are found together. People with this hemoglobin combination can have signs and symptoms ranging from mild anemia to severe thalassemia major.

### 5. HBB Gene Editing

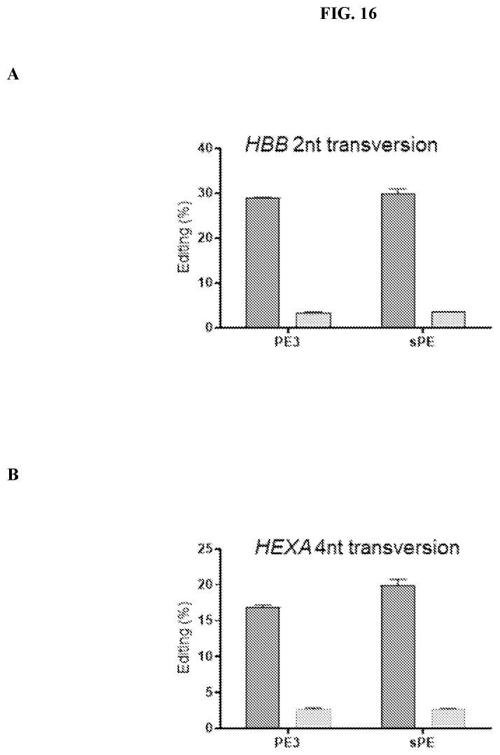

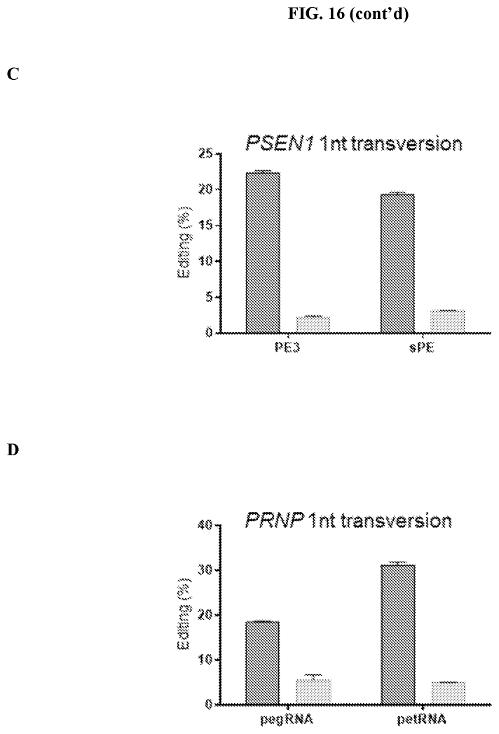

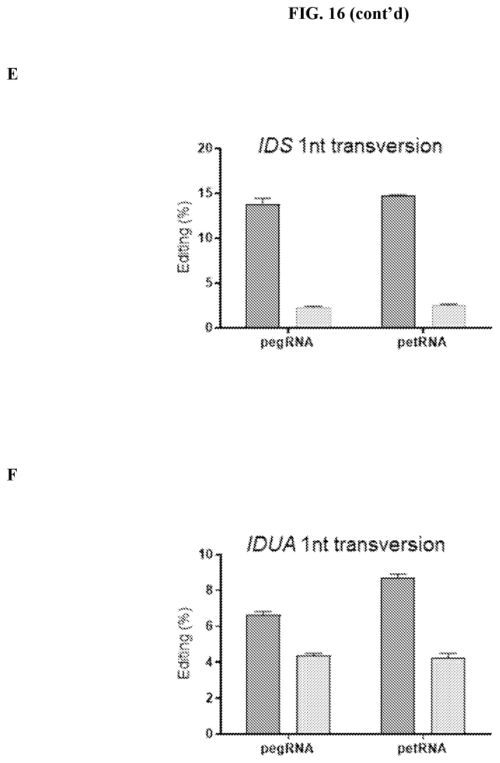

The data presented herein demonstrates equivalent HBB gene editing of a 2 nt transversion with a modular sPE construct comprising an HBB-specific pegRNA having the sequence of CAUGGUGCACCUGACUCCUGGUUUUAGAGCUAGAAAUAGCAAGUUAAAAUAAGGC UAGUCCGUUATCAACUUGAAAAAGUGGGACCGAGUCGGUCCAGACUUCUCUACAG GAGUCAGGUGCAC (SEQ ID NO:1) and a nicking guide sgRNA having the sequence of CCUUGAUACCAACCUGCCCAGUUUUAGAGCUAGAAAUAGCAAGUUAAAAUAAGGC UAGUCCGUUAUCAACUUGAAAAAGUGGCACCGAGUCGGUGC. See, FIG. 16A.

### B. Hexosaminidase, Subunit Alpha (HEXA) Genetic Diseases

The HEXA gene encodes a subunit of an enzyme called beta-hexosaminidase A. Specifically, a protein produced from a HEXA gene forms the alpha subunit of this enzyme. One alpha subunit joins with one beta subunit (produced from the HEXB gene) to form a functioning beta-hexosaminidase A enzyme.

Beta-hexosaminidase A is believed to play a role in brain and spinal cord function. Beta-hexosamindidase A is generally found in lysosomes, which are structures in cells that break down toxic substances and act as recycling centers. Within lysosomes, beta-hexosaminidase A forms part of a complex that breaks down a fatty substance called GM2 ganglioside found in cell membranes.

### 1. Tay-Sachs Disease

More than 210 mutations that cause Tay-Sachs disease have been identified in the HEXA gene. Tay-Sachs disease is a condition characterized by movement disorders, intellectual and developmental disability, and other neurological problems caused by the death of nerve cells in the central nervous system.

The HEXA gene variants that cause Tay-Sachs disease eliminate or severely reduce the activity of the enzyme beta-hexosaminidase A. This lack of enzyme activity prevents the enzyme from breaking down GM2 ganglioside. As a result, this substance builds up to toxic levels, particularly in neurons in the central nervous system. Progressive damage caused by the buildup of GM2 ganglioside leads to the destruction of these cells, which causes the signs and symptoms of Tay-Sachs disease.

Most of the known HEXA gene variants result in a completely nonfunctional version of beta-hexosaminidase A. These variants cause a severe form of Tay-Sachs disease, known as infantile Tay-Sachs disease, which appears in infancy. Other variants severely reduce but do not eliminate the activity of beta-hexosaminidase A. These genetic changes are responsible for less severe forms of Tay-Sachs disease, known as the juvenile and late-onset forms, which appear later in life.

### 2. HEXA Gene Editing

The data presented herein demonstrates equivalent HEXA gene editing of a 4 nt transversion with a modular sPE construct comprising an HEXA-specific pegRNA having the sequence of UACCUGAACCGUAUAUCCUAGUUUUAGAGCUAGAAAUAGCAAGUUAAAAUAAGGC UAGUCCGUUAUCAACUUGAAAAAGUGGGACCGAGUCGGUCCAGUCAGGGCCAUAG GAUAGAUAUACGGUUC (SEQ ID NO: 2) and a nicking guide sgRNA having the sequence of GCUUUCACCUUCAAAUGCCAGUUUUAGAGCUAGAAAUAGCAAGUUAAAAUAAGGC UAGUCCGUUAUCAACUUGAAAAAGUGGCACCGAGUCGGUGC. See, FIG. 16B.

### C. Presenilin 1 (PSEN1) Genetic Diseases

The PSEN1 gene encodes a protein called presenilin 1. This protein is a subunit of a gamma-(γ-) secretase complex. Presenilin 1 has proteolyic activity and cleaves other proteins into peptides.

The γ-secretase complex is located in the cell membrane where it cleaves many different proteins types of transmembrane proteins. This cleavage process participates in transmembrane signaling pathways that transmit biochemical messages from outside the cell into the nucleus. One of these pathways, known as Notch signaling, plays a role in the normal maturation and division of hair follicle cells and other types of skin cells. Notch signaling is also involved in normal immune system function.

The γ-secretase complex may be best known for its role in processing amyloid precursor protein (APP), which is made in the brain and other tissues. γ-secretase cuts APP into smaller peptides, including soluble amyloid precursor protein (sAPP) and several versions of amyloid-beta (β) peptide. Evidence suggests that sAPP has growth-promoting properties and may play a role in the formation of nerve cells (neurons) in the brain both before and after birth. Other functions of sAPP and amyloid-β peptide are under investigation.

### 1. Alzheimer Disease

More than 150 PSEN1 gene mutations have been identified in patients with early-onset Alzheimer disease, a degenerative brain condition that begins before age 65. Mutations in the PSEN1 gene are the most common cause of early-onset Alzheimer disease, accounting for up to 70 percent of cases.

Almost all PSEN1 gene mutations change single DNA nucleotides in a particular segment of the PSEN1 gene. These mutations result in the production of an abnormal presenilin 1 protein. Defective presenilin 1 interferes with the function of the γ-secretase complex, which alters the processing of APP and leads to the overproduction of a longer, toxic version of amyloid-β peptide. Copies of this protein fragment stick together and build up in the brain, forming clumps called amyloid plaques that are a characteristic feature of Alzheimer disease. A buildup of toxic amyloid-β peptide and the formation of amyloid plaques likely lead to the death of neurons and the progressive signs and symptoms of this disorder.

### 2. Hidradenitis Suppurativa

At least one mutation in the PSEN1 gene has been found to cause hidradenitis suppurativa, a chronic skin disease characterized by recurrent boil-like lumps (nodules) under the skin that develop in hair follicles. The nodules tend to become inflamed and painful, and they produce significant scarring as they heal.

The identified mutation deletes a single DNA nucleotide from the PSEN1 gene (e.g., 725delC). This genetic change reduces the amount of functional presenilin 1 produced in cells, so less of this protein is available to act as part of the γ-secretase complex. The resulting shortage of normal γ-secretase impairs cell signaling pathways, including Notch signaling. Although little is known about the mechanism, studies suggest that abnormal Notch signaling may promote the development of recurrent nodules in hair follicles and trigger inflammation in the skin.

Studies suggest that the PSEN1 gene mutation that causes hidradenitis suppurativa has a different effect on γ-secretase function than the mutations that cause early-onset Alzheimer disease. These differences may explain why no single PSEN1 gene mutation has been reported to cause the signs and symptoms of both diseases.

### 3. PSEN1 Gene Editing

The data presented herein demonstrates equivalent PSEN1 gene editing of a 1 nt transversion with a modular sPE construct comprising an PSEN1-specific pegRNA having the sequence of AAAGAGCAUGAUCACAUGCUGUUUUAGAGCUAGAAAUAGCAAGUUAAAAUAAGGC UAGUCCGUUAUCAACUUGAAAAAGUGGGACCGAGUCGGUCCAAAUAUGGCGUCAA GCAUGUGAUCAUGCUCU (SEQ ID NO: 3) and a nicking guide sgRNA having the sequence of UUAUCUAAUGGACGACCCCAGUUUUAGAGCUAGAAAUAGCAAGUUAAAAUAAGGC UAGUCCGUUAUCAACUUGAAAAAGUGGCACCGAGUCGGUGC. See, FIG. 16C.

### D. Prion Protein (PRNP) Genetic Diseases

The PRNP gene encodes a protein called prion protein (PrP), which is active in the brain and several other tissues. Although the precise function of this protein is unknown, researchers have proposed roles in several processes. These include, but are not limited to, the transport of copper into cells and/or protection of brain cells (neurons) from injury (neuroprotection). Studies have also suggested a role for PrP in the formation of synapses, which are the junctions between nerve cells (neurons) where cell-to-cell communication occurs.

Different forms of PrP have been identified. The normal version is often designated PrP^{C} to distinguish it from abnormal forms of the protein, which are generally designated PrP^{Sc}.

### 1. Huntington Disease-Like Syndrome

A particular type of mutation in the PRNP gene has been found to cause signs and symptoms that resemble those of Huntington disease, including uncontrolled movements, emotional problems, and loss of thinking ability. Researchers have proposed that this condition be called Huntington disease-like 1 (HDL1).

The PRNP mutations associated with HDL1 are believed located in an octapeptide repeat. This repeat encodes eight (8) amino acids and is normally repeated five times in the PRNP gene. In people with HDL1, this segment is repeated eleven or thirteen times. An increase in the size of the octapeptide repeat leads to the production of an abnormally long version of PrP. It is unclear how the abnormal protein damages and ultimately destroys neurons, leading to the characteristic features of HDL1.

### 2. Prion Disease

More than 30 mutations in the PRNP gene have been identified in people with familial forms of prion disease including, but not limited to, Creutzfeldt-Jakob disease (CJD), Gerstmann-Straussler-Scheinker syndrome (GSS), and/or fatal familial insomnia (FFI). The major features of these diseases include, but are not limited to, changes in memory, personality, and behavior; a decline in intellectual function (dementia); and abnormal movements, particularly difficulty with coordinating movements (ataxia). The signs and symptoms generally worsen over time, and can be fatal.

Some of the PRNP gene mutations that cause familial prion disease change single amino acids in PrP. Other mutations insert additional amino acids into the protein or result in an unusually short version of the protein. These changes alter the structure of PrP, leading to the production of an abnormally shaped protein, known as PrP^{Sc}, from one copy of the PRNP gene. In a process that is not fully understood, PrP^{Sc} can attach (bind) to PrP^{C} and promote its transformation into PrP^{Sc}. The abnormal protein builds up in the brain, forming clumps that damage or destroy neurons. The loss of these cells creates microscopic sponge-like holes (vacuoles) in the brain, which leads to the signs and symptoms of prion disease.

Several common variations (polymorphisms) in the PRNP gene have been identified that affect single amino acids in PrP. These polymorphisms do not cause prion disease, but they may affect a person's risk of developing these disorders. Studies have focused on the effects of a polymorphism at position 129 of PrP. At this position, people can have either the amino acid methionine (Met) or the amino acid valine (Val) (e.g., Met129Val or M129V). Because people inherit one copy of the PRNP gene from each parent, at position 129 an individual can receive methionine from both parents (Met/Met), valine from both parents (Val/Val), or methionine from one parent and valine from the other (Met/Val).

The Met129Val polymorphism appears to influence the risk of developing prion disease. Most affected individuals have the same amino acid at position 129 (Met/Met or Val/Val) instead of different amino acids (Met/Val). Having Met/Met at position 129 is also associated with an earlier age of onset and a more rapid worsening of the disease's signs and symptoms.

### 3. Wilson Disease

The PRNP Met129Val polymorphism has been reported to influence the onset of Wilson disease, an inherited disorder in which excessive amounts of copper accumulate in the body. While the primary cause of Wilson disease is an ATP7B gene mutation, symptoms of Wilson disease begin several years later in people who have Met/Met at position 129 in PrP as compared with those who have Met/Val or Val/Val. Other research findings indicate that this polymorphism may also affect the type of symptoms that develop in people with Wilson disease. Having Met/Met at position 129 appears to be associated with an increased occurrence of symptoms that affect the nervous system, particularly tremors.

### 4. PRNP Gene Editing

The data presented herein demonstrates equivalent PRNP gene editing of a 1 nt transversion with a pegRNA construct having the sequence of GCAGUGGUGGGGGGCCUUGGGUUUUAGAGCUAGAAAUAGCAAGUUAAAAUAAGG CUAGUCCGUUAUCAACUUGAAAAAGUGGGACCGAGUCGGUCCAUGUAGACGCCAA GGCCCCCCACC (SEQ ID NO: 4), an sgRNA construct having the sequence of GCAGUGGUGGGGGGCCUUGGGUUUUAGAGCUAGAAAUAGCAAGUUAAAAUAAGG CUAGUCCGUUAUCAACUUGAAAAAGUGGCACCGAGUCGGUGC, a petRNA construct having the sequence of AACCAUGCCGAGUGCGGCCGCAACUAAGCACAUGAGGAUCACCCAUGUGCAUGUA GACGCCAAGGCCCCCCACCAAAUUAACAGUGGCCGCGGUCGGCGUGGACUGUAG (SEQ ID NO: 5), and a nicking guide sgRNA having the sequence of GCAUGUUUUCACGAUAGUAAGUUUUAGAGCUAGAAAUAGCAAGUUAAAAUAAGG CUAGUCCGUUAUCAACUUGAAAAAGUGGCACCGAGUCGGUGC. See, FIG. 16D.

### E. Iduronate 2-Sulfatase (IDS) Gene Diseases

The IDS gene encodes an enzyme called iduronate 2-sulfatase (I2S), which degrades large sugar molecules called glycosaminoglycans (GAGs). Specifically, I2S removes a sulfate from sulfated alpha-L-iduronic acid, which is present in GAGs such as heparan sulfate and dermatan sulfate. I2S is located in lysosomes, compartments within cells that digest and recycle different types of molecules.

### 1. Mucopolysaccharidosis Type II

More than 300 mutations in the IDS gene have been found to cause mucopolysaccharidosis type II (MPS II). Mutations that change one nucleotide are the most common. All presently known mutations that cause MPS II reduce or completely eliminate the function of I2S. It usually cannot be determined whether a certain mutation will cause severe or mild MPS II; however, mutations that result in the complete absence of I2S cause the more severe form of the disorder.

Lack of I2S enzyme activity leads to the accumulation of heparan sulfate and dermatan sulfate within cells, specifically inside the lysosomes. The buildup of these GAGs increases the size of the lysosomes, which is why many tissues and organs are enlarged in MPS II. It is believed that the accumulated GAGs may also interfere with the functions of other proteins inside the lysosomes and disrupt the movement of molecules inside the cell.

### 4. IDS Gene Editing

The data presented herein demonstrates equivalent IDS gene editing of a 1 nt transversion with a pegRNA construct having the sequence of ACUGAGGGAUGUCUGAAGGCGUUUUAGAGCUAGAAAUAGCAAGUUAAAAUAAGG CUAGUCCGUUAUCAACUUGAAAAAGUGGGACCGAGUCGGUCCGCCAGUAUCCCUG GCCUUCAGACAUCCCU (SEQ ID NO: 6), an sgRNA construct having the sequence of ACUGAGGGAUGUCUGAAGGCGUUUUAGAGCUAGAAAUAGCAAGUUAAAAUAAGG CUAGUCCGUUAUCAACUUGAAAAAGUGGCACCGAGUCGGUGC, a petRNA construct having the sequence of AACCAUGCCGAGUGCGGCCGCAACUAAGCACAUGAGGAUCACCCAUGUGCGCCAG UAUCCCUGGCCUUCAGACAUCCCUAAAUUAACAGUGGCCGCGGUCGGCGUGGACU GUAG (SEQ ID NO: 7), and a nicking guide sgRNA having the sequence of GCAUUUUCGAUUCCGUGACUGUUUUAGAGCUAGAAAUAGCAAGUUAAAAUAAGGC UAGUCCGUUAUCAACUUGAAAAAGUGGCACCGAGUCGGUGC. See, FIG. 16E.

### F. Iduronidase, Alpha-L- (IDUA) Gene Diseases

The IDUA gene encodes an enzyme called alpha-L-iduronidase, which breaks down large sugar molecules called glycosaminoglycans (GAGs). Through a process called hydrolysis, alpha-L-iduronidase uses water molecules to break down unsulfated alpha-L-iduronic acid, which is present GAGs such as heparan sulfate and dermatan sulfate. Alpha-L-iduronidase is located in lysosomes, compartments within cells that digest and recycle different types of molecules.

### 1. Mucopolysaccharidosis Type I

More than 100 mutations in the IDUA gene have been found to cause mucopolysaccharidosis type I (MPS I). Mutations that change one DNA nucleotide are the most common. All presently known mutations that cause MPS I reduce or completely eliminate the function of alpha-L-iduronidase. It usually cannot be determined whether a certain mutation will cause severe or attenuated MPS I; however, people who do not produce any alpha-L-iduronidase have the severe form of this disorder.

The lack of alpha-L-iduronidase enzyme activity leads to the accumulation of heparan sulfate and dermatan sulfate within the lysosomes. The buildup of these GAGs increases the size of the lysosomes, which is why many tissues and organs are enlarged in MPS I. Researchers believe that the accumulated GAGs may also interfere with the functions of other proteins inside the lysosomes and disrupt the movement of molecules inside the cell.

### 2. IUDA Gene Editing

The data presented herein demonstrates equivalent IUDA gene editing of a 1 nt transversion with a pegRNA construct having the sequence of CCGCAGAUGAGGAGCAGCUCGUUUUAGAGCUAGAAAUAGCAAGUUAAAAUAAGGC UAGUCCGUUAUCAACUUGAAAAAGUGGGACCGAGUCGGUCCACACUUCGGCCUAG AGCUGCUCCUCAUC (SEQ ID NO: 8), an sgRNA construct having the sequence of CCGCAGAUGAGGAGCAGCUCGUUUUAGAGCUAGAAAUAGCAAGUUAAAAUAAGGC UAGUCCGUUAUCAACUUGAAAAAGUGGCACCGAGUCGGUGC, a petRNA construct having the sequence of AACCAUGCCGAGUGCGGCCGCAACUAAGCACAUGAGGAUCACCCAUGUGCACACU UCGGCCUAGAGCUGCUCCUCAUCAAAUUAACAGUGGCCGCGGUCGGCGUGGACUG UAG (SEQ ID NO: 5), and a nicking guide sgRNA having the sequence of GGCCGGGCCCUGGGGGCGGUGUUUUAGAGCUAGAAAUAGCAAGUUAAAAUAAGGC UAGUCCGUUAUCAACUUGAAAAAGUGGCACCGAGUCGGUGC. See, FIG. 16F.

### IV. Modular sPE System Pharmaceutical Delivery Systems

The present invention contemplates several delivery systems for sPE systems that provide for roughly uniform distribution, have controllable rates of release. A variety of different media are described below that are useful in creating such delivery systems. It is not intended that any one medium or carrier is limiting to the present invention. Note that any medium or carrier may be combined with another medium or carrier.

Carriers or mediums contemplated by this invention comprise a material selected from the group comprising gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcin-aldehyde type bioadhesives, polyacrylic acid and copolymers and block copolymers thereof.

One embodiment of the present invention contemplates an sPE delivery system further comprising a supplemental therapeutic agent.

### A. Ribonucleoprotein (RNP) Nucleotransfection

In one embodiment, the present invention contemplates sPE system mRNA delivery. Although it is not necessary to understand the mechanism of an invention, it is believed that delivery of two smaller modular PE mRNAs (e.g., a Cas9/RT mRNA and a pegRNA or petRNA) would improve overall stability and large scale manufacturing efficiency as opposed to full length split PE fusion constructs that are approximately 6-7 kb length. Commercial translation of a full length split PE fusion construct is also problematic due to its small size. Consequently, RNP compositions comprising sPE RNA systems (e.g., nSpy Cas9 RNA+MCP-fused reverse transcriptase) provides both manufacturing and clinical advantages. In one embodiment, an RNP composition comprising sPE RNA systems are administered using ribonucleotransfection.

To efficiently transport CRISPR-Cas into target tissues/cells require overcoming several extra- and intra-cellular barriers, therefore largely limiting the applications of CRISPR-based therapeutics in vivo. Suggested delivery platforms include, but are not limited to, plasmids, RNAs and ribonucleoproteins (RNPs).

RNPs are composed of a large Cas protein and a short gRNA. gRNA can bind to DNA via Watson-Crick base pairing or the Cas protein can be conjugated to polypeptides, proteins, and PEI. These features can also be used for loading RNP. In addition, RNP can be loaded via electrostatic interactions with positively charged materials due to its negative net charge. These positively charged materials can be cationic lipids, PEI, polypeptides, and metal-organic frameworks (MOFs). Vesicles from cells can also be used to deliver RNP. It has been reported that PEI can coat a complex of Cas9 RNP and DNA nanoclews for enhanced endosomal escape. PEI-coated DNA nanoclews were shown to efficiently transfect a Cas9 RNP targeting EGFP into U2OS cells for EGFP knockout in vitro. Furthermore, the PEI-coated DNA nanoclews could also disrupt EGFP in U2OS.EGFP xenograft tumors in vivo after intratumoral injection. Recently, a nanocapsule was developed for Cas9 RNP delivery. Due to the heterogeneous surface charges of RNP, the RNP was first coated with both cationic and anionic monomers via electrostatic interactions. An imidazole-containing monomer (e.g., glutathione (GSH)-degradable crosslinker) and, PEG can be absorbed to the surface of the RNP via hydrogen bonding and van der Waals interactions. Then, GSH-cleavable nanocapsules were formed around the RNP via in situ free-radical polymerization. In addition, targeting ligands, for example CPPs, can be added into the nanocapsule by conjugation to PEG. It was demonstrated that the GSH cleavable nanocapsule could protect Cas9 RNP in the endosome after cellular uptake and could be quickly cleaved by GSH after escape into the cytoplasm for subsequent genome editing. After local injection of Cas9 RNP nanocapsules, robust gene editing was observed in retinal pigment epithelium (RPE) and muscle. Because the net charge of RNP is negative, cationic liposomes or LNPs can be directly used for RNP transfection. It was demonstrated that the Cas9 protein (+22 net charges) can be rendered highly anionic by fusion to a negatively charged GFP (−30 net charges) or complexation with a gRNA. Alternatively, the positively charged PEI has also been developed for RNP delivery. For example, Cas9 RNP was loaded onto GO-PEG-PEI via physisorption and 7π-stacking interactions. Xu et al., “Rational designs of in vivo CRISPR-Cas delivery systems” Adv Drug Deliv Rev (2021).

RNP delivery for genome editing in live cells may be performed with Lipofectamine® RNAiMAX lipid transfection reagent and components of an sPE system. For example, pegRNAs/petRNAs are mixed with purified Cas9/RT proteins at an equimolar ratio in Opti-MEM™ to from an RNP complex (e.g, ˜10 min at room temperature). These RNPs can then be transfected into live cells using, for example, DMEM with 10% FBS. RNP nucleotransfection may be performed by electroporation using, for example, a Lonza 96-well Shuttle™ System (Lonza, Basel, Switzerland) optionally in the presence of Alt-R® Cas9 Electroporation Enhancer (Integrated DNA Technologies, Inc). Vakulskas et al., “A high-fidelity Cas9 mutant delivered as a ribonucleoprotein complex enables efficient gene editing in human haematopoietic stem and progenitor cells” Nat Med. 24(8): 1216-1224 (2018).

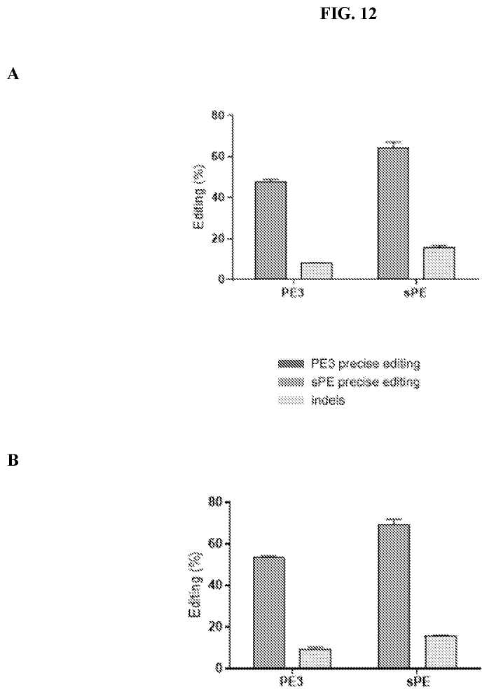

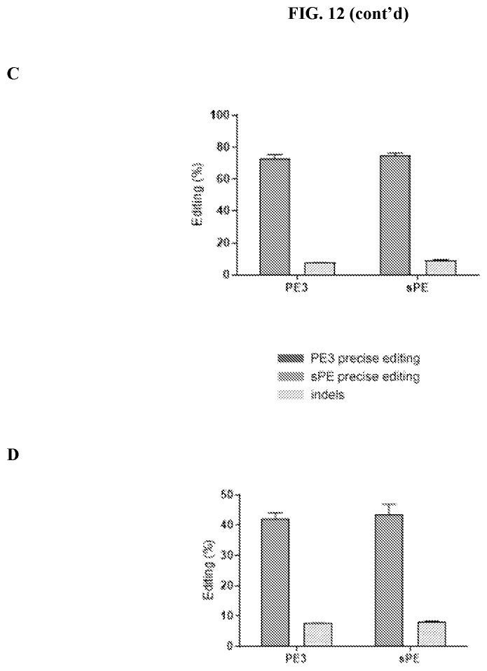

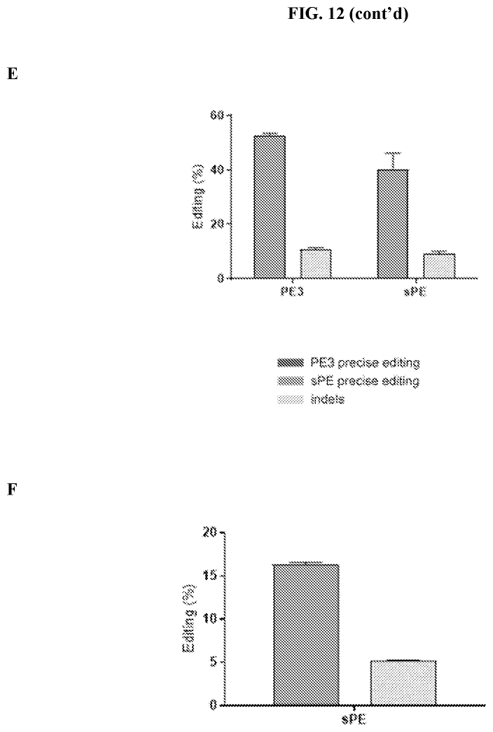

The prime editing efficiency of a number of genes was compared between PE3 and sPE in HEK293T cells using either conventional mRNA delivery or RNP-mediated nucleofection. For example, the genes included FANCF, VEGFA and HEK3. Conventional sPE-mRNA delivery was observed to result in improved gene editing efficiency of: FANCF 1 nt or a 3 nt transversion. See, FIGS. 12A and 12B. Conventional sPE-mRNA delivery was observed to result in equivalent gene editing efficiency of: i) VEGFA 3 nt transversion (See, FIG. 12C); and ii) HEK3 3 nt and 5 nt transversions (See, FIGS. 12D and 12E). RNP-mediated sPE-mRNA nucleotransfection was observed to result in a lower gene editing efficiency as opposed to sPE-mRNA delivery (e.g., ˜15% v.˜75%). Cf FIG. 12F v FIG. 12C).

### B. Microparticles

One embodiment of the present invention contemplates a medium comprising a microparticle. Preferably, microparticles comprise liposomes, nanoparticles, microspheres, nanospheres, microcapsules, and nanocapsules. Preferably, some microparticles contemplated by the present invention comprise poly(lactide-co-glycolide), aliphatic polyesters including, but not limited to, poly-glycolic acid and poly-lactic acid, hyaluronic acid, modified polysacchrides, chitosan, cellulose, dextran, polyurethanes, polyacrylic acids, psuedo-poly(amino acids), polyhydroxybutrate-related copolymers, polyanhydrides, polymethylmethacrylate, poly(ethylene oxide), lecithin and phospholipids.

### C. Liposomes

One embodiment of the present invention contemplates liposomes capable of attaching and releasing therapeutic agents described herein. Liposomes are microscopic spherical lipid bilayers surrounding an aqueous core that are made from amphiphilic molecules such as phospholipids. For example, a liposome may trap a therapeutic agent between the hydrophobic tails of the phospholipid micelle. Water soluble agents can be entrapped in the core and lipid-soluble agents can be dissolved in the shell-like bilayer. Liposomes have a special characteristic in that they enable water soluble and water insoluble chemicals to be used together in a medium without the use of surfactants or other emulsifiers. Liposomes can form spontaneously by forcefully mixing phosopholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life. One embodiment of the present invention contemplates an ultra high-shear technology to refine liposome production, resulting in stable, unilamellar (single layer) liposomes having specifically designed structural characteristics. These unique properties of liposomes, allow the simultaneous storage of normally immiscible compounds and the capability of their controlled release.

In some embodiments, the present invention contemplates cationic and anionic liposomes, as well as liposomes having neutral lipids. Preferably, cationic liposomes comprise negatively-charged materials by mixing the materials and fatty acid liposomal components and allowing them to charge-associate. Clearly, the choice of a cationic or anionic liposome depends upon the desired pH of the final liposome mixture. Examples of cationic liposomes include lipofectin, lipofectamine, and lipofectace.

One embodiment of the present invention contemplates a medium comprising liposomes that provide controlled release of at least one therapeutic agent. Preferably, liposomes that are capable of controlled release: i) are biodegradable and non-toxic; ii) carry both water and oil soluble compounds; iii) solubilize recalcitrant compounds; iv) prevent compound oxidation; v) promote protein stabilization; vi) control hydration; vii) control compound release by variations in bilayer composition such as, but not limited to, fatty acid chain length, fatty acid lipid composition, relative amounts of saturated and unsaturated fatty acids, and physical configuration; viii) have solvent dependency; iv) have pH-dependency and v) have temperature dependency.

The compositions of liposomes are broadly categorized into two classifications. Conventional liposomes are generally mixtures of stabilized natural lecithin (PC) that may comprise synthetic identical-chain phospholipids that may or may not contain glycolipids. Special liposomes may comprise: i) bipolar fatty acids; ii) the ability to attach antibodies for tissue-targeted therapies; iii) coated with materials such as, but not limited to lipoprotein and carbohydrate; iv) multiple encapsulation and v) emulsion compatibility.

Liposomes may be easily made in the laboratory by methods such as, but not limited to, sonication and vibration. Alternatively, compound-delivery liposomes are commercially available. For example, Collaborative Laboratories, Inc. are known to manufacture custom designed liposomes for specific delivery requirements.

### D. Microspheres, Microparticles and Microcapsules

Microspheres and microcapsules are useful due to their ability to maintain a generally uniform distribution, provide stable controlled compound release and are economical to produce and dispense. Preferably, an associated delivery gel or the compound-impregnated gel is clear or, alternatively, said gel is colored for easy visualization by medical personnel.

Microspheres are obtainable commercially (Prolease®, Alkerme's: Cambridge, Mass.). For example, a freeze dried medium comprising at least one therapeutic agent is homogenized in a suitable solvent and sprayed to manufacture microspheres in the range of 20 to 90 m. Techniques are then followed that maintain sustained release integrity during phases of purification, encapsulation and storage. Scott et al., Improving Protein Therapeutics With Sustained Release Formulations, Nature Biotechnology, Volume 16:153-157 (1998).

Modification of the microsphere composition by the use of biodegradable polymers can provide an ability to control the rate of therapeutic agent release. Miller et al., Degradation Rates of Oral Resorbable Implants {Polylactates and Polyglycolates: Rate Modification and Changes in PLA PGA Copolymer Ratios, J. Biomed. Mater. Res., Vol. I1:711-719 (1977).

Alternatively, a sustained or controlled release microsphere preparation is prepared using an in-water drying method, where an organic solvent solution of a biodegradable polymer metal salt is first prepared. Subsequently, a dissolved or dispersed medium of a therapeutic agent is added to the biodegradable polymer metal salt solution. The weight ratio of a therapeutic agent to the biodegradable polymer metal salt may for example be about 1:100000 to about 1:1, preferably about 1:20000 to about 1:500 and more preferably about 1:10000 to about 1:500. Next, the organic solvent solution containing the biodegradable polymer metal salt and therapeutic agent is poured into an aqueous phase to prepare an oil/water emulsion. The solvent in the oil phase is then evaporated off to provide microspheres. Finally, these microspheres are then recovered, washed and lyophilized. Thereafter, the microspheres may be heated under reduced pressure to remove the residual water and organic solvent.

Other methods useful in producing microspheres that are compatible with a biodegradable polymer metal salt and therapeutic agent mixture are: i) phase separation during a gradual addition of a coacervating agent; ii) an in-water drying method or phase separation method, where an antiflocculant is added to prevent particle agglomeration and iii) by a spray-drying method.

In one embodiment, the present invention contemplates a medium comprising a microsphere or microcapsule capable of delivering a controlled release of a therapeutic agent for a duration of approximately between 1 day and 6 months. In one embodiment, the microsphere or microparticle may be colored to allow the medical practitioner the ability to see the medium clearly as it is dispensed. In another embodiment, the microsphere or microcapsule may be clear. In another embodiment, the microsphere or microparticle is impregnated with a radio-opaque fluoroscopic dye.

Controlled release microcapsules may be produced by using known encapsulation techniques such as centrifugal extrusion, pan coating and air suspension. Such microspheres and/or microcapsules can be engineered to achieve desired release rates. For example, Oliosphere® (Macromed) is a controlled release microsphere system. These particular microsphere's are available in uniform sizes ranging between 5-500 μm and composed of biocompatible and biodegradable polymers. Specific polymer compositions of a microsphere can control the therapeutic agent release rate such that custom-designed microspheres are possible, including effective management of the burst effect. ProMaxx® (Epic Therapeutics, Inc.) is a protein-matrix delivery system. The system is aqueous in nature and is adaptable to standard pharmaceutical delivery models. In particular, ProMaxx® are bioerodible protein microspheres that deliver both small and macromolecular drugs, and may be customized regarding both microsphere size and desired release characteristics.

In one embodiment, a microsphere or microparticle comprises a pH sensitive encapsulation material that is stable at a pH less than the pH of the internal mesentery. The typical range in the internal mesentery is pH 7.6 to pH 7.2. Consequently, the microcapsules should be maintained at a pH of less than 7. However, if pH variability is expected, the pH sensitive material can be selected based on the different pH criteria needed for the dissolution of the microcapsules. The encapsulated compound, therefore, will be selected for the pH environment in which dissolution is desired and stored in a pH preselected to maintain stability. Examples of pH sensitive material useful as encapsulants are Eudragit® L-100 or S-100 (Rohm GMBH), hydroxypropyl methylcellulose phthalate, hydroxypropyl methylcellulose acetate succinate, polyvinyl acetate phthalate, cellulose acetate phthalate, and cellulose acetate trimellitate. In one embodiment, lipids comprise the inner coating of the microcapsules. In these compositions, these lipids may be, but are not limited to, partial esters of fatty acids and hexitiol anhydrides, and edible fats such as triglycerides. Lew C. W., Controlled-Release pH Sensitive Capsule And Adhesive System And Method. U.S. Pat. No. 5,364,634 (herein incorporated by reference).

In one embodiment, the present invention contemplates a microparticle comprising a gelatin, or other polymeric cation having a similar charge density to gelatin (i.e., poly-L-lysine) and is used as a complex to form a primary microparticle. A primary microparticle is produced as a mixture of the following composition: i) Gelatin (60 bloom, type A from porcine skin), ii) chondroitin 4-sulfate (0.005%-0.1%), iii) glutaraldehyde (25%, grade 1), and iv) 1-ethyl-3-(3-dimethylaminopropyl)-carbodiimide hydrochloride (EDC hydrochloride), and ultra-pure sucrose (Sigma Chemical Co., St. Louis, Mo.). The source of gelatin is not thought to be critical; it can be from bovine, porcine, human, or other animal source. Typically, the polymeric cation is between 19,000-30,000 daltons. Chondroitin sulfate is then added to the complex with sodium sulfate, or ethanol as a coacervation agent.

Following the formation of a microparticle, a therapeutic agent is directly bound to the surface of the microparticle or is indirectly attached using a “bridge” or “spacer”. The amino groups of the gelatin lysine groups are easily derivatized to provide sites for direct coupling of a compound. Alternatively, spacers (i.e., linking molecules and derivatizing moieties on targeting ligands) such as avidin-biotin are also useful to indirectly couple targeting ligands to the microparticles. Stability of the microparticle is controlled by the amount of glutaraldehyde-spacer crosslinking induced by the EDC hydrochloride. A controlled release medium is also empirically determined by the final density of glutaraldehyde-spacer crosslinks.

In one embodiment, the present invention contemplates microparticles formed by spray-drying a composition comprising fibrinogen or thrombin with a therapeutic agent. Preferably, these microparticles are soluble and the selected protein (i.e., fibrinogen or thrombin) creates the walls of the microparticles. Consequently, the therapeutic agents are incorporated within, and between, the protein walls of the microparticle. Heath et al., Microparticles And Their Use In Wound Therapy. U.S. Pat. No. 6,113,948 (herein incorporated by reference). Following the application of the microparticles to living tissue, the subsequent reaction between the fibrinogen and thrombin creates a tissue sealant thereby releasing the incorporated compound into the immediate surrounding area.

One having skill in the art will understand that the shape of the microspheres need not be exactly spherical; only as very small particles capable of being sprayed or spread into or onto a surgical site (i.e., either open or closed). In one embodiment, microparticles are comprised of a biocompatible and/or biodegradable material selected from the group consisting of polylactide, polyglycolide and copolymers of lactide/glycolide (PLGA), hyaluronic acid, modified polysaccharides and any other well known material.

### V. Modular sPE Administration Pharmaceutical Compositions

The present invention further provides pharmaceutical compositions comprising modular sPE systems as described herein. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the compound is administered in maintenance doses, ranging from 0.01 μg to 100 g per kg of body weight, once or more daily, to once every 20 years.

### Experimental

### Example I

### Plasmids

PegRNA expression plasmids were constructed by using a custom vector (BfuAI- and EcoRI173 digested). Liu, P. et al., “Improved prime editors enable pathogenic allele correction and cancer modelling in adult mice” Nat Commun 12 (2021). The gBlock fragments with or without MS2 sequences were synthesized by Integrated DNA Technologies (IDT), followed by Gibson assembly using Gibson Assembly® Master Mix (New England Biolabs, NEB). Colonies were selected and confirmed by Sanger sequencing using a commercial human U6 primer. Nicking sgRNA plasmids were generated by annealing oligos and inserting them into the pMD217 vector digested by BfuAI.

The MS2 bacteriophage coat protein (MCP) and the Moloney Murine Leukemia Virus Reverse Transcriptase (M-MLV RT) has been described elsewhere. Konermann et al., “Genome-scale transcriptional activation by an engineered CRISPR-Cas9 complex” Nature 517:583-588 (2015); and Peabody, D. S., “The RNA binding site of bacteriophage MS2 coat protein” EMBO 324 J 12:595-600 (1993). Briefly, the sequence of M-MLV Reverse transcriptase (RT) was derived from a PE2 construct via PCR. The vector was prepared by excising nCas9 through dual digestion of PE2 plasmids with NotI and KpnI. Two gBlock gene fragments were synthesized for fusing MCP and M-MLV RT partially together with either a 32-amino-acid linker or an NLS linker, followed by Gibson assembly. The ligation mixtures were transformed into competent HB101 cells. The colonies were selected and confirmed by Sanger sequencing.

The scFv-RT plasmids were constructed by replacing nCas9 with the scFv fragment in PE2. The scFv sequence was derived from Addgene #60904 via PCR21, followed by Gibson assembly. The 3×Flag-RT plasmid was constructed by excising nCas9. The 3×Flag sequence was derived from Addgene #80456 via PCR. The DNA fragments were assembled by Gibson assembly.

Gene fragments of alternative RTs along with their bridging fragments were synthesized by Genewiz. Prime editors with alternative RTs were constructed by Gibson Assembly with PE2 digested by EcoRI and BsmI. Split alternative RT plasmids were modified from the 3×Flag-RT plasmid by digesting with NotI and EcoRI followed by Gibson Assembly with appropriate gene fragments.

The ribozyme-flanked RNA expression plasmids were constructed by replacing the pegRNA sequence from the U6-driven plasmid using Gibson Assembly with synthesized ribozyme fragments and a fragment containing the transcript to be circularized flanked by the hairpin sequences for circularization.

Plasmids were purified by Miniprep Kit (QIAGEN) or ZymoPURE™ II Plasmids Miniprep Kit for in vitro experiments.

### Example II

Cell Culture, RNP and mRNA Delivery, and Genomic DNA Isolation

### Cell Culture Conditions

HEK293T cells (ATCC) were cultured in Dulbecco's Modified Eagle Medium with 10% FBS and 1% Penicillin/Streptomycin. The mCherry reporter line and GFP reporter lines have been described elsewhere. Wang et al., “sgBE: a structure-guided design of sgRNA architecture specifies base editing window and enables simultaneous conversion of cytosine and adenosine” Genome Biol 21:222 (2020). Cells were cultured at 37° C. and 5% CO₂.

mRNA Production

To construct the IVT templates, a CleanCap Reagent AG-compatible T7 promoter (TAATACGACTCACTATAAG) and a 5′ UTR were inserted at the 5′ of the Kozak sequence of the coding sequence in the mammalian expression vectors for nCas9, MMLV-RT and PE2. Additionally, a 3′ UTR, a 110-nt poly(A) tract and a restriction site (BsmBI) were inserted after the stop codon. Plasmids were completely linearized using BsmBI (New England Biolabs) before being used in the IVT, which was carried out at 37° C. using the HiScrib T7 High Yield RNA Synthesis Kit (New England Biolabs) with the addition of CleanCap Reagent AG (Trilink Biotechnologies) and with a 100% replacement of UTP by N1-Methylpseudo-UTP (Trilink Biotechnologies). The reaction was terminated after 4 h by a 15-min incubation with DNase I (New England Biolabs) added. The RNA was then purified using Monarch RNA Cleanup Kit (New England Biolabs).

### RT Protein Production

For bacterial expression of MMLV-RT protein, the coding sequence was cloned into a pET28a vector and transformed into BL21 (DE3) Rosetta competent cells (Novagen) and selected on Luria broth (LB) agar plates containing kanamycin (Kan) and chloramphenicol (Cam). One liter of LB+Kan+Cam media was inoculated at 37° C. by 4 ml of overnight culture from a single colony. The culture was induced with 1 mM IPTG at OD₆₀₀ 0.8 for 3 h at 37° C. The pellet was washed with washed with 1×PBS and snap-frozen using liquid nitrogen.

For purification of MMLV-RT protein, the cell pellet was resuspended in Lysis Buffer (50 mM Tris-HCl, pH 8.0, 100 mM NaCl, 10 mM imidazole, 1 mM DTT and 0.1% Triton X-100) and incubated on ice for 30 min with lysozyme added to a final concentration of 1 mg/ml. The cells were lysed by using an EpiShea Probe Sonicator (Active Motif) and cleared by centrifuge at 20,000 g for 30 min at 4° C., before being loaded on a column with Ni-NTA resin pre-equilibrated with Wash Buffer I (50 mM Tris-HCl, pH 8.0, 500 mM NaCl, 10 mM imidazole, 1 mM DTT and 0.1% Triton X-100). The resin was washed by 10 column volume (CV) Wash Buffer I and 10 CV Wash Buffer II (50 mM Tris-HCl, pH 8.0, 100 mM NaCl, 10 mM imidazole and 1 mM DTT), before the protein was eluted using elution buffer (50 mM Tris-HCl, pH 8.0, 100 mM NaCl, 250 mM imidazole and 1 mM DTT). The eluate was dialyzed against Buffer A (50 mM Tris-HCl, pH 8.0, 100 mM NaCl, 0.1 mM EDTA and 5 mM DTT) overnight before being loaded onto a HiTrap SP HP cation exchange chromatography column (Cytiva). The column was washed with CV Buffer A before the protein was eluted with a 0-100% gradient of Buffer B (50 mM Tris-HCl, pH 8.0, 1 M NaCl, 0.1 mM EDTA and 5 mM DTT). The peak fractions were pooled and buffer exchanged with Storage Buffer (50 mM Tris-HCl, pH 8.0, 100 mM NaCl, 0.1 mM EDTA and 5 mM DTT and 30% glycerol).

RNP and mRNA Nucleofection

For nucleofections of mRNA and RNP, the Neon electroporation system was used. For mRNA nucleofection, 1 μg of each mRNA, 120 picomoles of pegRNA (Integrated DNA Technologies), 40 picomoles of nicking guide (Integrated DNA Technologies) and 50,000 HEK293T cells were mixed in buffer R and electroporated using 10 μL Neon tips, with electroporation parameters as follows: 1150 V, 20 ms, 2 pulses. After electroporation, cells were plated in pre-warmed 48-well plates with DMEM media containing 10% FBS and incubated for 72 h before analysis. For RNP nucleofection, same conditions were used except that the mRNAs were replaced by 61 picomoles of nCas9 protein (Alt-R S.p. Cas9 H840A Nickase V3, purchased from Integrated DNA Technologies) and 150 picomoles of NLS-containing MMLV-RT protein produced in-house.

### Plasmid Lipofection

Transfection of the plasmids in HEK293T cells were carried out according to the manufacturer's instructions for the Lipofectamine 3000 reagent (Invitrogen, #L3000015). Briefly, 1×10⁵ cells were seeded per well in a 12-well plate overnight. Cells were transfected using 3 μl Lipofectamine 3000 and P3000 (2 μl/μg DNA). For each well, 330 ng pegRNA, 110 ng nicking sgRNA, and 1 μg PE2 plasmids were used. The same amount of plasmids were used for modular sPE groups. After 72 hours post-transfection, cells were harvested and lysed using 100 μl Quick extraction buffer (Lucigene). Subsequently, the lysis was incubated on a PCR machine with 65° C. incubation for 15 min, 98° C. for 5 min.

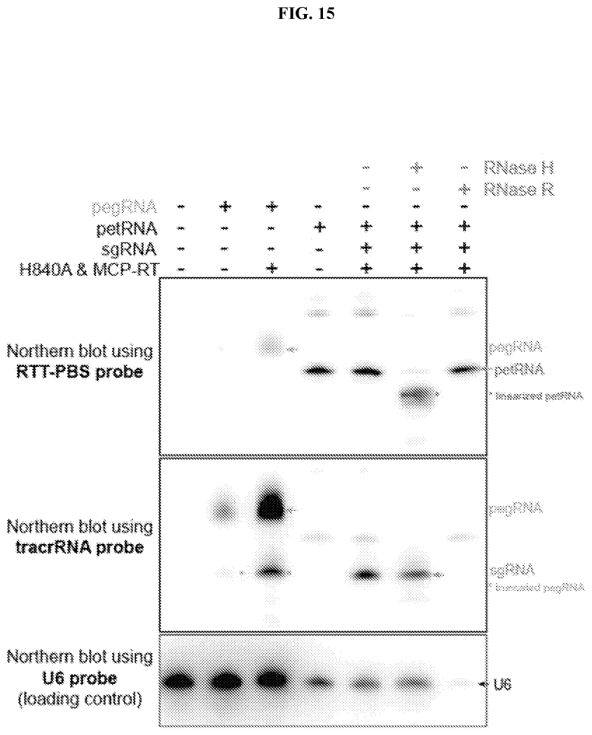

The integrity and abundance of the transiently expressed pegRNAs and petRNAs were assessed in Northern blots with multiple probes 72 h post-transfection in HEK293T cells. Probes were labeled using 32P-ATP and T4 PNK. Total RNAs containing the petRNA were loaded 10 times less than those without to allow better comparison between levels of pegRNA and petRNA when using the RTT-PBS probe. See, FIG. 15. Putative cleavage products are labeled by asterisks. For the last sample, RNase H and RNase R were used to demonstrate a circular nature of a petRNA.

### Mouse DNA Isolation Technique

Mouse genomic DNA was isolated using PureLink Genomic DNA Mini Kit (Thermo Fisher) according to the manufacturer's protocol.

### Example III

### Sanger Sequencing and Analysis

PCR amplification was performed around the target locus using Phusion Flash PCR Master Mix (Thermo Fisher) and specific primers. Sanger sequencing was performed by GENEWIZ (South Plainfield, NJ). The results were quantified using EditR22.

### Example IV

### Deep Sequencing and Data Analysis

Sequencing library preparation was done as described previously. Anzalone et al., “Search-and-replace genome editing without double-strand breaks or donor DNA”. Nature 576:149-157 (2019). Briefly, for the first round of PCR, specific primers carrying Illumina forward and reverse adapters were used for amplifying the genomic sites of interest with Phusion Hot Start II PCR Master Mix.

For the second-round PCR, primers containing unique Illumina barcodes were used. PCR reactions were performed as follows: 98° C. for 10 s, then (98° C. for 1 s, 55° C. for 5 s, and 72° C. for 6 s) for 20 cycles, followed by 72° C. extensions for 2 min as a final extension. The DNA products of second-round PCR were collected and purified by gel purification using the QIAquick Gel Extraction Kit (Qiagen). DNA concentration was determined by Qubit dsDNA HS Assay. Subsequently, the library was sequenced on an Illumina MiniSeq following the manufacturer's protocols.

MiniSeq sequencing reads were demultiplexed by bcl2fastq (Illumina). Prime editing efficiency was determined by aligning amplicon reads to a reference sequence using CRISPResso. The frequencies of precise editing and indels were quantified with CRISPResso2 in the standard model.

### Example V

### Flow Cytometry Analysis

Flow cytometry analysis was performed on day 3 after transfection. mCherry or GFP reporter lines were harvested after PBS washing and 0.25% trypsin digestion, followed by re-centrifuging at 300×g for 5 min followed by resuspension in PBS with 2% FBS. The proportions of GFP and/or mCherry-positive cells were quantified using flow cytometry (MACSQuant VYB). Data were analysed by FlowJo v10 software.

### Example VI

### Animals

All mice studies were approved by the Institutional Animal Care and Use Committee (IACUC) at UMass medical school. All plasmids were prepared using an Endo-Free-Maxi kit (Qiagen) and delivered by hydrodynamic tail-vein injection. For cancer model generation, eight-week-old 246 FVB/NJ mice (Strain #001800) were injected with 30 μg PE2 or split Cas9 nickase and RT, 15 μg pegRNA, 15 μg sgRNA nicking, 5 μg pT3 EF1a-MYC (Addgene #92046) and 1 μg CMV SB10 (Addgene #24551) via the tail vein.

### Example VII

### Histology and Immunohistochemistry

The procedure of IHC staining has been described²⁴. Briefly, livers were fixed with 4% formalin overnight, embedded with paraffin, and sectioned at 5 μM, followed by hematoxylin and eosin (H&E) staining for pathology. Liver sections were de-waxed, rehydrated, and stained according to previous immunohistochemistry protocols²⁵. The following antibody was used: β-CATENIN (BD, 610154, 1:200). The images were captured by Leica DMi8 microscopy.

### Example VIII

### Gene Editing Protocol of Disease Modifying Loci

Cell transfection is according to the manufacturer's instructions for the Lipofectamine 3000 reagent (Invitrogen, #L3000015). Briefly, 1×105 cells were seeded per well in a 12-well plate overnight. Cells were transfected using 3 μl Lipofectamine 3000 and P3000 (2 μl/μg DNA). For each well, 330 ng pegRNA, 110 ng nicking sgRNA, and 1 μg PE2 plasmids were used. The same amount of plasmids were used for sPE groups. After 72 hours post-transfection, cells were harvested and lysed using 100 μl Quick extraction buffer (Lucigene). Subsequently, the lysis was incubated on a PCR machine with 65° C. incubation for 15 min, 98° C. for 5 min.

### Example IX

In Vivo Gene Editing with Modular sPE

### AAV Vector Package, Production, and Injection

AAV vectors (AAV8 capsids) were packaged and produced at the Viral Vector Core of the Horae Gene Therapy Center, University of Massachusetts Medical School. Virus titers were measured by gel electrophoresis followed by silver staining and ddPCR.

For AAV injection, 1×10¹² GC AAV-Cas9^{H840A} and 1λ10¹² GC AAV-U6-pegRNA-U6-sgRNA-M-MLV were resuspended in 200 μL 0.9% sodium chloride and administered via tail vein injection

1. A modular prime editing system, comprising:
i) a first ribonucleic acid (RNA) encoding a Cas9 nickase protein and a reverse transcriptase protein; and
ii) a second RNA encoding a component selected from the group consisting of a single guide RNA, a primer binding site and a reverse transcriptase template (RTT);
wherein said first RNA and said second RNA are not attached or tethered.

2. The system of claim 1, wherein said second RNA comprises a prime editor guide RNA (pegRNA).

3. The system of claim 1, wherein said second RNA comprises a prime editor template RNA (petRNA).

4. The system of claim 3, wherein said petRNA is circularized.

5. The system of claim 3, wherein said petRNA is linear.

6. The system of claim 1, wherein said first RNA is less than 4.5 kB.

7. The system of claim 1, wherein said second RNA is less than 4.5 kB.

8. The system of claim 1, wherein the combination of said first and said second RNA is less then 4.5 kB.

9. A composition, comprising:
a) a ribonucleic acid (RNA) delivery system; and
b) a modular prime editing system, comprising:
i) a first ribonucleic acid (RNA) encoding a Cas9 nickase protein and a reverse transcriptase protein; and
ii) a second RNA comprising a component selected from the group consisting of a single guide RNA, a primer binding site and a reverse transcriptase template (RTT),
wherein said first RNA and said second RNA are not attached or tethered.

10. The composition of claim 9, wherein said second RNA comprises a prime editor guide RNA (pegRNA).

11. The composition of claim 9, wherein said second RNA comprises a prime editor template RNA (petRNA).

12. The composition of claim 11, wherein said petRNA is circularized.

13. The composition of claim 11, wherein said petRNA is linear.

14. The composition of claim 9, wherein said first RNA is less than 4.5 kB.

15. The composition of claim 9, wherein said second RNA is less than 4.5 kB.

16. The composition of claim 9, wherein the combination of said first and said second RNA is less than 4.5 kB.

17. The composition of claim 9, wherein said RNA delivery system is an adeno-associated virus (AAV) delivery system.

18. The composition of claim 17, wherein said first RNA and said second RNA are encased within said AAV.

19. The composition of claim 9, wherein said RNA delivery system is a ribonucleoprotein delivery system.

20. The composition of claim 9, wherein said RNA delivery system is a microparticle system or a liposome system.

21-47. (canceled)


SEQUENCE LISTING (46 sequences)

SEQ ID NO: 1 - RNA, 123 nt, synthetic construct
  source 1..123: mol_type = other RNA
catggtgcacctgactcctggttttagagctagaaatagcaagttaaaataaggctagtccgttatcaacttgaaaaagtgggaccgagtcggtccagacttctctacaggagtcaggtgcac

SEQ ID NO: 2 - RNA, 126 nt, synthetic construct
  source 1..126: mol_type = other RNA
tacctgaaccgtatatcctagttttagagctagaaatagcaagttaaaataaggctagtccgttatcaacttgaaaaagtgggaccgagtcggtccagtcagggccataggatagatatacggttc

SEQ ID NO: 3 - RNA, 127 nt, synthetic construct
  source 1..127: mol_type = other RNA
aaagagcatgatcacatgctgttttagagctagaaatagcaagttaaaataaggctagtccgttatcaacttgaaaaagtgggaccgagtcggtccaaatatggcgtcaagcatgtgatcatgctct

SEQ ID NO: 4 - RNA, 120 nt, synthetic construct
  source 1..120: mol_type = other RNA
gcagtggtggggggccttgggttttagagctagaaatagcaagttaaaataaggctagtccgttatcaacttgaaaaagtgggaccgagtcggtccatgtagacgccaaggccccccacc

SEQ ID NO: 5 - RNA, 109 nt, synthetic construct
  source 1..109: mol_type = other RNA
aaccatgccgagtgcggccgcaactaagcacatgaggatcacccatgtgcatgtagacgccaaggccccccaccaaattaacagtggccgcggtcggcgtggactgtag

SEQ ID NO: 6 - RNA, 125 nt, synthetic construct
  source 1..125: mol_type = other RNA
actgagggatgtctgaaggcgttttagagctagaaatagcaagttaaaataaggctagtccgttatcaacttgaaaaagtgggaccgagtcggtccgccagtatccctggccttcagacatccct

SEQ ID NO: 7 - RNA, 114 nt, synthetic construct
  source 1..114: mol_type = other RNA
aaccatgccgagtgcggccgcaactaagcacatgaggatcacccatgtgcgccagtatccctggccttcagacatccctaaattaacagtggccgcggtcggcgtggactgtag

SEQ ID NO: 8 - RNA, 124 nt, synthetic construct
  source 1..124: mol_type = other RNA
ccgcagatgaggagcagctcgttttagagctagaaatagcaagttaaaataaggctagtccgttatcaacttgaaaaagtgggaccgagtcggtccacacttcggcctagagctgctcctcatc

SEQ ID NO: 9 - RNA, 96 nt, synthetic construct
  source 1..96: mol_type = other RNA
ccttgataccaacctgcccagttttagagctagaaatagcaagttaaaataaggctagtccgttatcaacttgaaaaagtggcaccgagtcggtgc

SEQ ID NO: 10 - RNA, 96 nt, synthetic construct
  source 1..96: mol_type = other RNA
gctttcaccttcaaatgccagttttagagctagaaatagcaagttaaaataaggctagtccgttatcaacttgaaaaagtggcaccgagtcggtgc

SEQ ID NO: 11 - RNA, 96 nt, synthetic construct
  source 1..96: mol_type = other RNA
ttatctaatggacgaccccagttttagagctagaaatagcaagttaaaataaggctagtccgttatcaacttgaaaaagtggcaccgagtcggtgc

SEQ ID NO: 12 - RNA, 96 nt, synthetic construct
  source 1..96: mol_type = other RNA
gcagtggtggggggccttgggttttagagctagaaatagcaagttaaaataaggctagtccgttatcaacttgaaaaagtggcaccgagtcggtgc

SEQ ID NO: 13 - RNA, 96 nt, synthetic construct
  source 1..96: mol_type = other RNA
gcatgttttcacgatagtaagttttagagctagaaatagcaagttaaaataaggctagtccgttatcaacttgaaaaagtggcaccgagtcggtgc

SEQ ID NO: 14 - RNA, 96 nt, synthetic construct
  source 1..96: mol_type = other RNA
actgagggatgtctgaaggcgttttagagctagaaatagcaagttaaaataaggctagtccgttatcaacttgaaaaagtggcaccgagtcggtgc

SEQ ID NO: 15 - RNA, 96 nt, synthetic construct
  source 1..96: mol_type = other RNA
gcattttcgattccgtgactgttttagagctagaaatagcaagttaaaataaggctagtccgttatcaacttgaaaaagtggcaccgagtcggtgc

SEQ ID NO: 16 - RNA, 96 nt, synthetic construct
  source 1..96: mol_type = other RNA
ccgcagatgaggagcagctcgttttagagctagaaatagcaagttaaaataaggctagtccgttatcaacttgaaaaagtggcaccgagtcggtgc

SEQ ID NO: 17 - RNA, 96 nt, synthetic construct
  source 1..96: mol_type = other RNA
ggccgggccctgggggcggtgttttagagctagaaatagcaagttaaaataaggctagtccgttatcaacttgaaaaagtggcaccgagtcggtgc

SEQ ID NO: 18 - DNA, 19 bp, synthetic construct
  source 1..19: mol_type = other DNA
taatacgactcactataag

SEQ ID NO: 19 - RNA, 113 nt, synthetic construct
  source 1..113: mol_type = other RNA
aaccatgccgagtgcggccgcaactaagcacatgaggatcacccatgtgcacacttcggcctagagctgctcctcatcaaattaacagtggccgcggtcggcgtggactgtag

SEQ ID NO: 20 - DNA, 16 bp, synthetic construct
  source 1..16: mol_type = other DNA
tacgagggcactcaaa

SEQ ID NO: 21 - DNA, 13 bp, synthetic construct
  source 1..13: mol_type = other DNA
ccgccaagctgaa

SEQ ID NO: 22 - DNA, 15 bp, synthetic construct
  source 1..15: mol_type = other DNA
taccctcctcctcct

SEQ ID NO: 23 - DNA, 12 bp, synthetic construct
  source 1..12: mol_type = other DNA
ccctcaagtcct

SEQ ID NO: 24 - DNA, 16 bp, synthetic construct
  source 1..16: mol_type = other DNA
tacgagggcactcaaa

SEQ ID NO: 25 - DNA, 13 bp, synthetic construct
  source 1..13: mol_type = other DNA
cctcctcctccta

SEQ ID NO: 26 - DNA, 16 bp, synthetic construct
  source 1..16: mol_type = other DNA
taccctcctcctcctc

SEQ ID NO: 27 - DNA, 13 bp, synthetic construct
  source 1..13: mol_type = other DNA
ccgccaagctgaa

SEQ ID NO: 28 - DNA, 27 bp, synthetic construct
  source 1..27: mol_type = other DNA
  misc_feature 23: note = The residue at this position can be either G or T
cccagactgagcacgtgatggcagagg

SEQ ID NO: 29 - DNA, 16 bp, synthetic construct
  source 1..16: mol_type = other DNA
gcagcacctggatcgc

SEQ ID NO: 30 - DNA, 26 bp, synthetic construct
  source 1..26: mol_type = other DNA
gctccttccctgagtggcaagggcaa

SEQ ID NO: 31 - RNA, 23 nt, synthetic construct
  source 1..23: mol_type = other RNA
gctcctctgagtggcaagggcaa

SEQ ID NO: 32 - DNA, 28 bp, synthetic construct
  source 1..28: mol_type = other DNA
gctcctctgagtggcaagggcaaccctg

SEQ ID NO: 33 - DNA, 31 bp, synthetic construct
  source 1..31: mol_type = other DNA
gctccttccctgagtggcaagggcaaccctg

SEQ ID NO: 34 - DNA, 28 bp, synthetic construct
  source 1..28: mol_type = other DNA
gctcctctgagtggcaagggcaacccag

SEQ ID NO: 35 - DNA, 28 bp, synthetic construct
  source 1..28: mol_type = other DNA
gctcctctgagtggcaagggcaaccctg

SEQ ID NO: 36 - DNA, 31 bp, synthetic construct; identical to SEQ ID NO: 33
  source 1..31: mol_type = other DNA

SEQ ID NO: 37 - DNA, 28 bp, synthetic construct
  source 1..28: mol_type = other DNA
gctcctctgagtggcaagggcaacccag

SEQ ID NO: 38 - DNA, 45 bp, synthetic construct
  source 1..45: mol_type = other DNA
ggaagtagggccttcgcgcacctcatggaatcccttctgcagcac

SEQ ID NO: 39 - DNA, 41 bp, synthetic construct
  source 1..41: mol_type = other DNA
ttttcgggatggtcctcatgaacgactggagcagtaatgcc

SEQ ID NO: 40 - DNA, 41 bp, synthetic construct
  source 1..41: mol_type = other DNA
ttttcgggatggtcctcataaacgactggagcagtaatgcc

SEQ ID NO: 41 - DNA, 41 bp, synthetic construct; identical to SEQ ID NO: 39
  source 1..41: mol_type = other DNA

SEQ ID NO: 42 - DNA, 41 bp, synthetic construct; identical to SEQ ID NO: 39
  source 1..41: mol_type = other DNA

SEQ ID NO: 43 - DNA, 41 bp, synthetic construct
  source 1..41: mol_type = other DNA
ttttcgggatggtcctcatgaacgactggagcggtaatgcc

SEQ ID NO: 44 - DNA, 41 bp, synthetic construct
  source 1..41: mol_type = other DNA
ttttcgggatggtcctcatgaccgactggagcagtaatgcc

SEQ ID NO: 45 - DNA, 41 bp, synthetic construct; identical to SEQ ID NO: 39
  source 1..41: mol_type = other DNA

SEQ ID NO: 46 - DNA, 41 bp, synthetic construct; identical to SEQ ID NO: 40
  source 1..41: mol_type = other DNA